(19) United States
(12) Patent Application Publication
(10) Pub. No.: US 2014/0335033 A1
(43) Pub. Date: Nov. 13, 2014

(54) Consumer Products Comprising Silane-Modified Oils

(71) Applicant: The Procter & Gamble Company, Cincinnati, OH (US)

(72) Inventors: Rajan Keshav PANANDIKER, West Chester, OH (US); Beth Ann SCHUBERT, Maineville, OH (US); John August WOS, Mason, OH (US); Luke Andrew ZANNONI, West Chester, OH (US); Nathan Ray Whitely, Liberty, OH (US)

(73) Assignee: The Procter & Gamble Company, Cincinnati, OH (US)

(21) Appl. No.: 14/272,744

(22) Filed: May 8, 2014

Related U.S. Application Data
(60) Provisional application No. 61/821,818, filed on May 10, 2013.

(51) Int. Cl. A61K 8/92; A61Q 15/00; A61Q 3/02; A61Q 9/04; C11D 3/16; A61Q 19/10; A61Q 5/02; A61Q 19/00; A61Q 5/00

(52) U.S. Cl. CPC A61K 8/92; A61Q 19/00; A61K 8/922; A61Q 15/00; A61Q 3/02; A61Q 5/00; C11D 3/162; A61Q 19/10; A61Q 5/02; A61Q 9/04
USPC 424/61; 514/772; 514/786; 514/772.4; 514/781; 514/778; 514/777; 424/65; 424/70.12; 514/770; 510/466; 510/138; 510/130; 510/122; 510/276; 510/228; 510/235; 510/295; 8/161; 512/4

(57) ABSTRACT

A consumer product comprises silane-modified oil comprising a hydrocarbon chain selected from the group consisting of: a saturated oil, an unsaturated oil, and mixtures thereof; and at least one hydrolysable silyl group covalently bonded to the hydrocarbon chain. The consumer product further comprises a perfume.

### FIELD OF THE INVENTION

Consumer products comprising silane-modified oils, particles comprising silane-modified oils, and/or gels comprising silane-modified oils and further comprising a perfume. Certain of the consumer products can include cosmetics, personal beauty care, shaving care, household care, fabric care compositions and the like.

### BACKGROUND OF THE INVENTION

Silicone elastomers have been widely used to enhance the performance of consumer products such as cosmetics, personal care, household care, and fabric care compositions. Silicone elastomers are generally obtained by a crosslinking hydrosilylation reaction of an SiH polysiloxane with another polysiloxane containing an unsaturated hydrocarbon substituent, such as a vinyl functional polysiloxane, or by crosslinking an SiH polysiloxane with a hydrocarbon diene. The silicone elastomers may be formed in the presence of a carrier fluid, such as a volatile silicone, resulting in a gelled composition. Alternatively, the silicone elastomer may be formed at higher solids content, subsequently sheared and admixed with a carrier fluid to also create gels or paste like compositions.

Derivative silicone elastomers have also been commercialized. Since they are easily functionalized, silicone elastomers can be customized to provide a variety of benefits. This versatility is one reason why silicone elastomers are so prevalent in consumer product compositions.

Despite their many benefits, silicone elastomers can pose formulation challenges when combined with various other materials included in consumer products. Blend performance depends not only upon the properties of the individual components but also upon the blend morphology and the interfacial properties existing between the different blend components.

For example, silicone elastomers do not always exhibit good compatibility with organic or hydrocarbon (e.g. non-silicone) oils. Phase incompatibility can result in immiscible, phase-separated blends due to high interfacial tension between the silicone elastomers and the non-silicone oils. In the case of cosmetic foundations, for instance, silicone elastomers may not be able to incorporate the amount of non-silicone oil desired in the product, and/or the oil may exude from the elastomer in the finished product, resulting in an unsatisfactory consumer use experience.

Silicone oils and similar components are commonly used in making a wide variety of consumer products. In recent years, as manufacturers and consumers have gained a greater awareness of environmental and sustainability concerns, the demand for materials having lower levels of silicone has grown significantly.

Further, materials that might deliver benefits similar to those of silicone oils may be hydrocarbon materials that may include a degree of unsaturation. Hydrocarbon oils in general may have disadvantages in that they are frequently odorous and can impart an undesirable base-odor to products comprising them. Further, hydrocarbon oils bearing unsaturated moieties can be susceptible to oxidation, which can further impart an undesirable base-odor to products as said oxidation can lead to rancidity over time.

Accordingly, it would be desirable to provide materials that can deliver the performance advantages of silicone elastomers as well as the environmental advantages of materials having significant non-silicone fractions, and that do not have an undesirable base-odor perceived by the consumer. Such materials should be stable and suitable for use in a wide range of consumer product applications.

### SUMMARY OF THE INVENTION

The present invention provides consumer product compositions comprising silane-modified oils, particles comprising silane-modified oils, and/or gels comprising silane-modified oils and further comprising a perfume. These oils and/or particles and/or gels can be used to provide a variety of desired performance benefits in various consumer product forms.

The invention provides additional aspects directed to such silane-modified oils, particles comprising silane-modified oils, and gels comprising silane-modified oils and further comprising a perfume. The silane-modified oils and/or particles comprising silane-modified oils and/or gels comprising silane-modified oils can comprise an added benefit agent; alternatively, the silane-modified oils and/or particles comprising silane-modified oils and/or gels comprising silane-modified oils can function as, and therefore be considered, a benefit agent.

In one aspect, the invention provides consumer product compositions comprising a silane-modified oil comprising: (a) a hydrocarbon chain, and (b) a hydrolysable silyl group covalently bonded to said hydrocarbon chain. In a particular aspect, the silane-modified oil comprises:
(i) at least one hydrocarbon chain selected from the group consisting of: a saturated oil, an unsaturated oil, and mixtures thereof; and
(ii) at least one hydrolysable silyl group covalently bonded to the hydrocarbon chain.
   and a perfume.

In another aspect, the invention provides consumer product compositions comprising particles comprising silane-modified oils and a perfume. The particles comprise: (1) a particle core having an interfacial surface; and (2) a silane-modified oil moiety attached to said interfacial surface. The particle can additionally comprise an optional polymer having a property. The silane-modified oil and optionally the polymer are attached to the interfacial surface of the particle core at different locations on the interfacial surface. In some aspects, the particle comprises two or more than two polymers and/or properties.

In another aspect, the invention provides consumer product compositions comprising gels comprising silane-modified oils and a perfume. The gel comprises the reaction product of (a) a silane-modified oil, and (b) water, where at least some of the oil's hydrolysable silyl groups have been condensed, forming covalent intermolecular siloxane crosslinks between the oil molecules and/or other cross-linking moieties in the consumer product composition.

In a particular aspect, the gels comprising silane-modified oils comprise the reaction product of:
(a) a silane-modified oil comprising:
   (i) a hydrocarbon chain selected from the group consisting of: a saturated oil, an unsaturated oil, and mixtures thereof; and
   (ii) a hydrolysable silyl group covalently bonded to the hydrocarbon chain; and
(b) perfume
(c) water;
(d) at least one additional component comprising at least one hydroxyl moiety where:
   (i) at least some of the hydrolysable silyl groups of the silane-modified oil have been condensed, thereby forming covalent intermolecular siloxane crosslinks between the silicon-based moieties of the silane-modified oil molecules in the crosslinked silane-modified oil; and
   (ii) the crosslinked silane-modified oil is sufficiently crosslinked with the intermolecular siloxane crosslinks to form a gel.

The invention also provides a method for treating a surface, comprising: (a) applying at least one of the consumer product compositions comprising the silane-modified oil and a perfume to the surface, and (b) optionally applying water to said surface. In another aspect, the method comprises: (a) applying the consumer product compositions comprising the silane-modified, oil-based gel to a surface, and (b) optionally applying water to said surface.

In a particular development, the consumer product comprises a delivery device having at least a first chamber and optionally second chamber. The first chamber comprises the silane-modified oil and optionally a non-aqueous solvent or carrier, while the optional second chamber comprises water.

Additional features of the disclosure may become apparent to those skilled in the art from a review of the following detailed description, taken in conjunction with the drawings, examples, and appended claims.

### BRIEF DESCRIPTION OF THE DRAWINGS

FIG. 1 illustrates grafting and crosslinking reactions associated with unsaturated triglyceride soy oil and unsaturated hydrolysable silane in one aspect of the present invention.

FIG. 2 illustrates generally a silane-modified oil bonded to the surface of a particle. An organo-functional silanol oil is shown attached to a silica surface.

FIG. 3 illustrates generally multiple silane-modified oils bonded to the surface of a particle. An organo-functional silanol oil is shown attached to a silica surface.

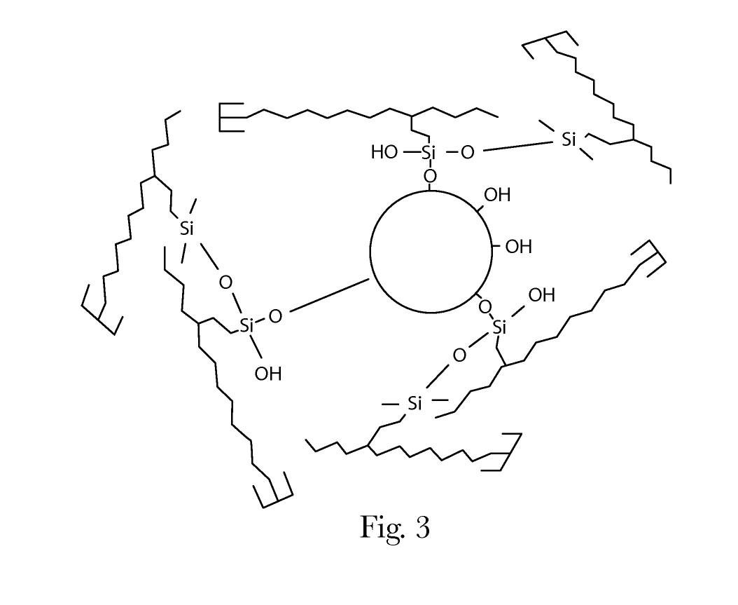

FIG. 4 illustrates a gel comprising a silane-modified oil and a hydroxy-functional inorganic particle and a hydroxyl-functional organic species.

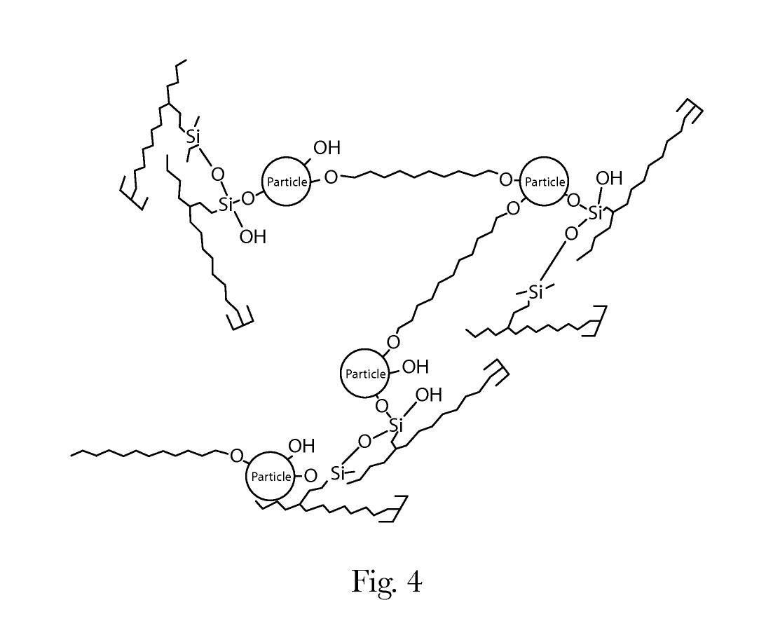

FIG. 5 illustrates a gel comprising a silane-modified oil and a hydroxy-functional organic species.

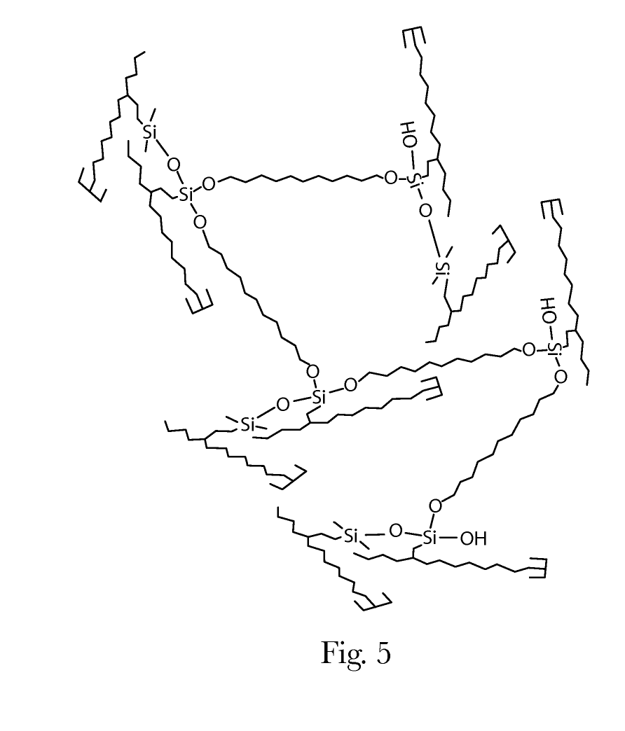

FIG. 6 illustrates a gel comprising a silane-modified oil and a hydroxy-functional inorganic particle.

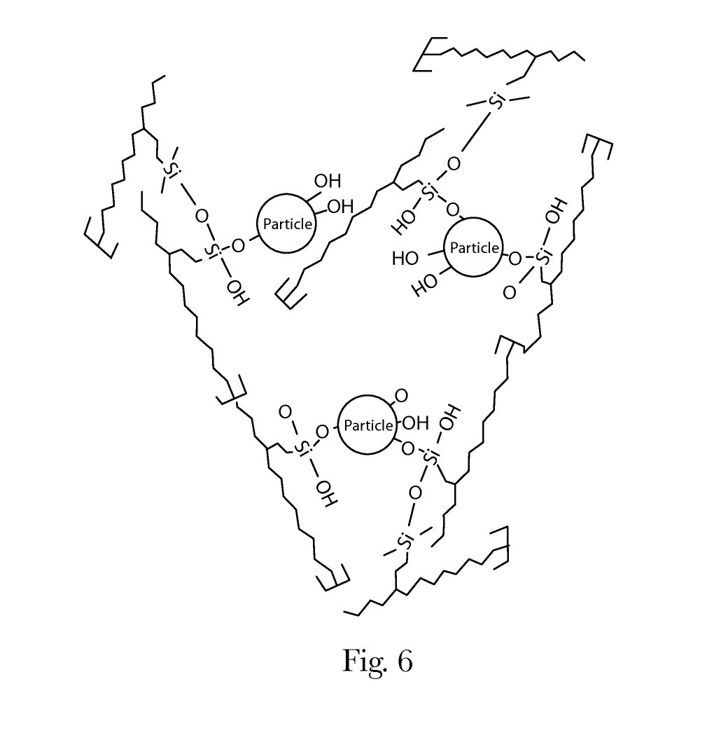

### DETAILED DESCRIPTION OF THE INVENTION

The present invention provides consumer product compositions comprising silane-modified oils, particles comprising silane-modified oils, and/or gels comprising silane-modified oils and further comprising a perfume. These oils and/or particles and/or gels can be used to provide a variety of desired performance benefits in various consumer product forms.

The invention provides additional aspects directed to such silane-modified oils, particles comprising silane-modified oils, and gels comprising silane-modified oil and further comprising a perfume. The silane-modified oils and/or particles comprising silane-modified oils and/or gels comprising silane-modified oils can comprise an added benefit agent; alternatively, the silane-modified oils and/or particles comprising silane-modified oils and/or gels comprising silane-modified oils can function as, and therefore be considered, a benefit agent.

In one aspect, the invention provides consumer product compositions comprising a silane-modified oil comprising: (a) a hydrocarbon chain, and (b) a hydrolysable silyl group covalently bonded to said hydrocarbon chain. In a particular aspect, the silane-modified oil comprises:
(i) at least one hydrocarbon chain selected from the group consisting of: a saturated oil, an unsaturated oil, and mixtures thereof; and
(ii) at least one hydrolysable silyl group covalently bonded to the hydrocarbon chain.
   and a perfume.

It has been found that that the silane-modified oils of the present invention are generally odorous and can be inappropriate for use in consumer products without mitigation of the off-odors generally associated with said silane modified oils and their feed-stock ingredients. Further, many of the reaction mechanisms used to generate silane modified oils require that the starting feedstock oil comprise unsaturated hydrocarbon moieties as the reaction-sights for the illation. These unsaturated hydrocarbon moieties can further negative impact the off-odors associated with these materials in that unsaturated hydrocarbon moieties can contribute to further oxidation of the oil over time, leading to rancidity. This previously unappreciated problem is addressed by the present invention by combining the silane-modified oil and a perfume to form the consumer product of the present invention.

In another aspect, the invention provides consumer product compositions comprising particles comprising silane-modified oils and a perfume. The particles comprise: (1) a particle core having an interfacial surface; and (2) a silane-modified oil moiety attached to said interfacial surface. The particle can additionally comprise an optional polymer having a property. The silane-modified oil and optionally the polymer are attached to the interfacial surface of the particle core at different locations on the interfacial surface. In some aspects, the particle comprises two or more than two polymers and/or properties.

In another aspect, the invention provides consumer product compositions comprising gels comprising silane-modified oils and a perfume. The gel comprises the reaction product of (a) a silane-modified oil, and (b) water, where at least some of the oil's hydrolysable silyl groups have been condensed, forming covalent intermolecular siloxane crosslinks between the oil molecules and/or other cross-linking moieties in the consumer product composition.

In a particular aspect, the gels comprising silane-modified oils comprise the reaction product of:
(a) a silane-modified oil comprising:
   (i) a hydrocarbon chain selected from the group consisting of: a saturated oil, an unsaturated oil, and mixtures thereof; and
   (ii) a hydrolysable silyl group covalently bonded to the hydrocarbon chain; and
(b) perfume
(c) water;
(d) at least one additional component comprising at least one hydroxyl moiety where:
   (i) at least some of the hydrolysable silyl groups of the silane-modified oil have been condensed, thereby forming covalent intermolecular siloxane crosslinks between the silicon-based moieties of the silane-modified oil molecules in the crosslinked silane-modified oil; and
   (ii) the crosslinked silane-modified oil is sufficiently crosslinked with the intermolecular siloxane crosslinks to form a gel.

In one aspect, the at least one additional component comprising at least one hydroxyl moiety can be selected from the group consisting of hydroxyl functionalized inorganic particles, hydroxyl functionalized organic species, and combinations thereof. Examples of suitable hydroxyl functionalized inorganic particles include metal oxides such as silica, titania, alumina, metallocene, and zeolite. Examples of hydroxyl functionalized organic species include oligosaccharides and polysaccharides and derivatives such as cellulose, guar, starch, cyclodextrin, hydroxypropyl guar, hydroxypropyl cellulose, guar hydroxypropyltrimonium chloride, polyquarternium-10, dimethiconol, hydroxyl terminated polybutadiene, polyethylene oxide, polypropylene oxide, and poly(tetramethylene ether)glycol. In a particular aspect, the hydroxyl functionalized species comprises multiple hydroxyl functions such that a bridge is formed between bonding sites on multiple silane-modified oils, thereby creating a gel.

The invention also provides a method for treating a surface, comprising: (a) applying at least one of the consumer product compositions comprising the silane-modified oil and a perfume to the surface, and (b) optionally applying water to said surface. In another aspect, the method comprises: (a) applying the consumer product compositions comprising the silane-modified, oil-based gel to a surface, and (b) optionally applying water to said surface.

The compositions and methods of the present invention are useful in treating surfaces such as fabric, textiles, leather, non-wovens or woven substrates, fibers, carpet, upholstery, glass, ceramic, skin, hair, fingernails, stone, masonry, wood, plastic, paper, cardboard, metal, packaging or a packaging component.

In a particular development, the consumer product comprises a delivery device having at least a first chamber and optionally second chamber. The first chamber comprises the silane-modified oil and optionally a non-aqueous solvent or carrier, while the optional second chamber comprises water. Either chamber may comprise the perfume.

As used herein, “oil” means any hydrocarbon-based material, including room temperature solids and room-temperature liquids. Oils include mono-, di-, and tri-glycerides, as well as fatty acids or their esters or aldehydes. Oils also include hydrocarbons, including hydrocarbons, aromatic hydrocarbons, and hydrocarbons containing both aliphatic and aromatic moieties. As used herein, “oils” also include hydrocarbon-based polymers, including polyvinyl polymers and their derivatives. Further, “oils” include linear, branched, or cross-linked polymers. In particular, the polymers includes polymers produced from one or more ethylenically unsaturated monomers. For purposes of the present invention, the backbone of a polymer produced from one or more ethylenically unsaturated monomers is considered to be a hydrocarbon chain (to which the hydrolyzable silyl group is covalently bonded thereto).

As used herein, “unsaturated oil” means an oil comprising at least one unsaturated hydrocarbon chain per molecule of the unsaturated oil. Unsaturated oils include mono-, di-, and tri-glycerides, as well as unsaturated fatty acids or their esters. Unsaturated oils also include unsaturated hydrocarbon chains. Unsaturated oils can be naturally unsaturated, or they can be manufactured from other materials (e.g., saturated oils) as is known in the art. For purposes of the present invention, the unsaturated backbone of a polymer produced from one or more ethylenically unsaturated monomers is considered to be an unsaturated hydrocarbon chain (to which the hydrolyzable silyl group is covalently bonded thereto).

As used herein, “saturated oil” means an oil that does not comprise any unsaturated hydrocarbon chains in the oil molecule. Saturated oils include mono-, di-, and tri-glycerides, as well as saturated fatty acids or their esters. Saturated oils also include saturated hydrocarbon chains. Saturated oils can be naturally saturated, or they can be manufactured from other materials (e.g., unsaturated oils) as is known in the art. For purposes of the present invention, the saturated backbone of a polymer produced from one or more ethylenically unsaturated monomers is considered to be a saturated hydrocarbon chain (to which the hydrolyzable silyl group is covalently bonded thereto).

As used herein “perfume” means a material that comprises one or more perfume raw materials and which provides a scent and/or decreases a malodor. It would be understood by one of ordinary skill in the art that a single perfume raw material can also provide a scent and/or decrease a malodor.

As used herein “preservative” means any substance that is added to the consumer product composition to prevent decomposition by microbial growth or by undesirable chemical changes. Preservatives may be naturally occurring or synthetically manufactured.

As used herein, “particulate benefit agent” means any ingredient that imparts a benefit in use where the ingredient is a solid at room temperature and not dissolved in the product.

As used herein, articles such as “a” and “an” when used in a claim, are understood to mean one or more of what is claimed or described.

As used herein, the term “solid” includes granular, powder, bar and tablet product forms.

As used herein, the term “fluid” includes liquid, gel, paste and gas product forms.

As used herein, the term “situs” includes paper products, fabrics, garments, hard surfaces, hair and skin.

As used herein, the terms “include”, “includes” and “including” are meant to be non-limiting.

Unless specified otherwise, all molecular weights are given in Daltons.

As used herein, the term “hydrocarbon polymer radical” means a polymeric radical comprising only carbon and hydrogen.

As used herein the term “siloxyl residue” means a polydimethylsiloxane moiety.

As used herein, “substituted” means that the organic composition or radical to which the term is applied is: (a) made unsaturated by the elimination of elements or radical; or (b) at least one hydrogen in the compound or radical is replaced with a moiety containing one or more (i) carbon, (ii) oxygen, (iii) sulfur, (iv) nitrogen or (v) halogen atoms; or (c) both (a) and (b).

Moieties that may replace hydrogen as described in (b) immediately above, which contain only carbon and hydrogen atoms are all hydrocarbon moieties including, but not limited to, alkyl, alkenyl, alkynyl, alkyldienyl, cycloalkyl, phenyl, alkyl phenyl, naphthyl, anthryl, phenanthryl, fluoryl, steroid groups, and combinations of these groups with each other and with polyvalent hydrocarbon groups such as alkylene, alkylidene and alkylidyne groups. Moieties containing oxygen atoms that may replace hydrogen as described in (b) immediately above include hydroxy, acyl or keto, ether, epoxy, carboxy, and ester containing groups. Moieties containing sulfur atoms that may replace hydrogen as described in (b) immediately above include the sulfur-containing acids and acid ester groups, thioether groups, mercapto groups and thioketo groups.

Moieties containing nitrogen atoms that may replace hydrogen as described in (b) immediately above include amino groups, the nitro group, azo groups, ammonium groups, amide groups, azido groups, isocyanate groups, cyano groups and nitrile groups. Specific non-limiting examples of such nitrogen containing groups are: —NHCH₃, —NH₂, —NH₃+, —CH₂CONH₂, —CH₂CON₃, —CH₂CH₂CH═NOH, —CN, —CH(CH₃)CH₂NCO, —CH₂NCO, —Nphi, -phi N═Nphi OH, and ≡N.

Moieties containing halogen atoms that may replace hydrogen as described in (b) immediately above include chloro, bromo, fluoro, iodo groups and any of the moieties previously described where a hydrogen or a pendant alkyl group is substituted by a halo group to form a stable substituted moiety. Specific non-limiting examples of such halogen containing groups are: —(CH₂)₃COCl, -phi F₅, -phi Cl, —CF₃, and —CH₂phi Br.

It is understood that any of the above moieties that may replace hydrogen as described in (b) can be substituted into each other in either a monovalent substitution or by loss of hydrogen in a polyvalent substitution to form another monovalent moiety that can replace hydrogen in the organic compound or radical.

As used herein “phi” or “ph” represents a phenyl ring.

As used herein, the nomenclature SiO“n”/2 represents the ratio of oxygen and silicon atoms. For example, SiO1/2 means that one atom oxygen is shared between two Si atoms. Likewise SiO2/2 means that two oxygen atoms are shared between two Si atoms and SiO3/2 means that three oxygen atoms are shared are shared between two Si atoms.

Unless otherwise noted, all component or composition levels are in reference to the active portion of that component or composition, and are exclusive of impurities, for example, residual solvents or by-products, which may be present in commercially available sources of such components or compositions.

All percentages and ratios are calculated by weight unless otherwise indicated. All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

### Consumer Product Compositions

The present application provides consumer products such as care agents comprising silane-modified oils, and/or gels comprising silane-modified oils, and/or particles comprising silane-modified oils. The silane modified oils can be incorporated into the consumer product compositions in any suitable form, depending upon desired end-use properties. For example, silane-modified oils can be pre-crosslinked to create Si—O—Si bonds. In one aspect, this crosslinking takes place between the silane-modified-oil and another material having hydroxyl groups (e.g. Si—OH groups selected from silica or siloxanes).

Compositions of the present invention can provide benefits such as softness, hand, anti-wrinkle, hair conditioning/frizz control, color protection, enhanced shine, increased spreadability, skin feel, and rheology modification (thickening), repellency, etc.

As used herein “consumer product” means baby care, personal care, fabric & home care, family care (e.g., facial tissues, paper towels), feminine care, health care, and like products generally intended to be used or consumed in the form in which it is sold. Such products include but are not limited to diapers, bibs, wipes; products for and/or methods relating to treating hair (human, dog, and/or cat), including, bleaching, coloring, dyeing, conditioning, shampooing, styling; deodorants and antiperspirants; personal cleansing; cosmetics; skin care including application of creams, lotions, and other topically applied products for consumer use including fine fragrances; and shaving products, products for and/or methods relating to treating fabrics, hard surfaces and any other surfaces in the area of fabric and home care, including: air care including air fresheners and scent delivery systems, car care, dishwashing, fabric conditioning (including softening and/or freshening), laundry detergency, laundry and rinse additive and/or care, hard surface cleaning and/or treatment including floor and toilet bowl cleaners, and other cleaning for consumer or institutional use; products and/or methods relating to bath tissue, facial tissue, paper handkerchiefs, and/or paper towels; tampons, and feminine napkins.

As used herein, the terms “consumer product” and “consumer product composition” are used interchangeably.

The compositions of the present invention can advantageously be used in cleaning and/or treatment compositions. As used herein, the term “cleaning and/or treatment composition” is a subset of consumer products that includes, unless otherwise indicated, beauty care, fabric & home care products. Such products include, but are not limited to, products for treating hair (human, dog, and/or cat), including, bleaching, coloring, dyeing, conditioning, shampooing, styling; deodorants and antiperspirants; personal cleansing; cosmetics; skin care including application of creams, lotions, and other topically applied products for consumer use including fine fragrances; and shaving products, products for treating fabrics, hard surfaces and any other surfaces in the area of fabric and home care, including: air care including air fresheners and scent delivery systems, car care, dishwashing, fabric conditioning (including softening and/or freshening), laundry detergency, laundry and rinse additive and/or care, hard surface cleaning and/or treatment including floor and toilet bowl cleaners, granular or powder-form all-purpose or “heavy-duty” washing agents, especially cleaning detergents; liquid, gel or paste-form all-purpose washing agents, especially the so-called heavy-duty liquid types; liquid fine-fabric detergents; hand dishwashing agents or light duty dishwashing agents, especially those of the high-foaming type; machine dishwashing agents, including the various tablet, granular, liquid and rinse-aid types for household and institutional use; liquid cleaning and disinfecting agents, including antibacterial hand-wash types, cleaning bars, mouthwashes, denture cleaners, dentifrice, car or carpet shampoos, bathroom cleaners including toilet bowl cleaners; hair shampoos and hair-rinses; shower gels, fine fragrances and foam baths and metal cleaners; as well as cleaning auxiliaries such as bleach additives and “stain-stick” or pre-treat types, substrate-laden products such as dryer added sheets, dry and wetted wipes and pads, nonwoven substrates, and sponges; as well as sprays and mists all for consumer or/and institutional use.

The compositions of the present invention can advantageously be used in fabric and/or hard surface cleaning and/or treatment compositions. As used herein, the term “fabric and/or hard surface cleaning and/or treatment composition” is a subset of cleaning and treatment compositions that includes, unless otherwise indicated, granular or powder-form all-purpose or “heavy-duty” washing agents, especially cleaning detergents; liquid, gel or paste-form all-purpose washing agents, especially the so-called heavy-duty liquid types; liquid fine-fabric detergents; hand dishwashing agents or light duty dishwashing agents, especially those of the high-foaming type; machine dishwashing agents, including the various tablet, granular, liquid and rinse-aid types for household and institutional use; liquid cleaning and disinfecting agents, including antibacterial hand-wash types, cleaning bars, car or carpet shampoos, bathroom cleaners including toilet bowl cleaners; and metal cleaners, fabric conditioning products including softening and/or freshening that may be in liquid, solid and/or dryer sheet form; as well as cleaning auxiliaries such as bleach additives and “stain-stick” or pre-treat types, substrate-laden products such as dryer added sheets, dry and wetted wipes and pads, nonwoven substrates, and sponges; as well as sprays and mists. All of such products which were applicable may be in standard, concentrated or even highly concentrated form even to the extent that such products may in certain aspect be non-aqueous.

The compositions of the present invention can advantageously be used in household polishes and cleaners for floors and countertops. They enhance shine, spread easily and do not chemically react with surface materials. The care agents in fabric softeners help preserve “newness” because of their softening properties, and their elasticity helps smooth out wrinkles. The care agents can also enhance shoe cleaning and polishing products.

The compositions of the present invention can advantageously be used to treat substrate-type products such as nonwoven fabric or sanitary tissue products. Non-limiting examples of consumer products of the present invention include absorbent articles selected from the group consisting of towels, towelettes, surface-cleaning wipes, fabric cleaning wipes, skin cleansing wipes, make-up removal wipes, applicator wipes, car cleaning wipes, lens cleaning wipes, packaging materials, cleaning wipes, dusting wipes, packing materials, disposable garments, disposable surgical or medical garments, bandages, paper-towels, toilet tissues, facial wipes, and wound dressings, baby diapers, training pants, adult incontinence articles, feminine protection articles, bed pads, and incontinent pads. In one aspect the absorbent article comprises a topsheet, backsheet or a barrier cuff treated with a composition of the present invention.

Substrates treated with compositions of the present invention can be useful in treating surfaces by contacting the treated substrate with the surface to be treated. In one aspect, said treated substrate may be a nonwoven fabric. In another aspect, said treated substrate may comprise a portion of an absorbent article.

In one aspect, the treated substrate is treated with less than 1 gram per square meter (gsm), or from 0.01-10 gsm, or from 0.01-5 gsm, or from 0.01-2 gsm of the composition of the composition of the present invention after said article is dried.

The composition of the present invention can be applied to the substrate by any of a number of means known to one of ordinary skill in the art. In one aspect the composition as applied to the substrate comprises a carrier selected from the group consisting of water, ethanol, solvents, isopropanol, surfactant, emulsifier, and combinations thereof.

### Silane-Modified Oils

A silane-modified oil according to the disclosure includes (a) a hydrocarbon chain selected from the group consisting of: a saturated oil, an unsaturated oil, and mixtures thereof; and (b) at least one hydrolysable silyl group covalently bonded to the hydrocarbon chain. The hydrolysable silyl group is generally covalently bonded to the hydrocarbon chain at an internal carbon position along the length of the chain, and not at a terminal carbon (e.g., a carbon at the chain end opposing an ester/acid group in a fatty acid/triglyceride).

The silane-modified oil can have any desired degree of unsaturation or can be fully saturated. The degree of unsaturation or saturation can be modified by one skilled in the art using any suitable process. Further, the hydrocarbon chain can be hydrogenated or dehydrogenated before, during, or after the hydrolysable silyl group is covalently bonded onto it, depending upon preference and the particular hydrogenation or dehydrogenation process used.

In one aspect, a process for forming the silane-modified oil according to the disclosure includes reacting an unsaturated oil with an unsaturated hydrolysable silane in the presence of a free radical initiator. The reaction thus forms a silane-modified oil having hydrolysable silyl groups covalently bonded to the unsaturated oil molecules. The resulting silane-modified oil can have any degree of silylation desirable for the specific product application. In one aspect, the silane-modified oil can comprise fewer than 1.2 hydrolysable silyl groups covalently bonded, on average, per molecule of silane-modified oil, preferably fewer than 1.0 hydrolysable silyl groups covalently bonded, on average, per molecule of silane-modified oil, preferably fewer than 0.8 hydrolysable silyl groups covalently bonded, on average, per molecule of silane-modified oil. In another aspect, the silane-modified oil can comprise more than 1.2 hydrolysable silyl groups covalently bonded, on average, per molecule of silane-modified oil, preferably more than 1.5 hydrolysable silyl groups covalently bonded, on average, per molecule of silane-modified oil, preferably more than 2.0 hydrolysable silyl groups covalently bonded, on average, per molecule of silane-modified oil. In another aspect the silane-modified oil can comprise from about 0.7 to about 5.0 hydrolysable silyl groups covalently bonded, on average, per molecule of silane-modified oil, preferably from about 0.7 to about 2.4 hydrolysable silyl groups covalently bonded, on average, per molecule of silane-modified oil, preferably from about 0.7 to about 1.6 hydrolysable silyl groups covalently bonded, on average, per molecule of silane-modified oil. In another aspect, the silane-modified oil can comprise more than 5.0 hydrolysable silyl groups covalently bonded, on average, per molecule of silane-modified oil.

The silane-modified oil may be purified prior to compounding into the consumer product of the present invention. Said purification may take any form of purification know to one of ordinary skill in the art. In one aspect, the silane modified oil is purified by removal of residual reagents, preferably residual reagents comprising silicon atoms. In one aspect, the purification comprises evaporation of residual reagent, preferably under vacuum and/or at a temperature above ambient temperature (e.g. 21° C.). In one aspect the purified silane-modified oil comprises less than about 10% residual reagent comprising at least one silicon atom, preferably less than about 5% residual reagent comprising at least one silicon atom, preferably less than about 1% residual reagent comprising at least one silicon atom, preferably less than about 0.1% residual reagent comprising at least one silicon atom.

Also disclosed is a process for crosslinking the silane-modified oil. The process includes crosslinking the silane-modified oil with water, thereby hydrolyzing and condensing the hydrolysable silyl groups to form covalent intermolecular siloxane crosslinks in the silane-modified oil. In one aspect, the silane-modified oil can be provided in a mixture with a crosslinking catalyst (e.g., titanium catalyst, tin catalyst).

In one aspect, the unsaturated oil can be derived from triglycerides comprised of fatty acid ester groups that collectively comprise at least one site of alkenyl unsaturation (e.g., at least one unsaturated hydrocarbon chain per molecule of unsaturated oil; generally not including silicone oils, alkoxy-terminated (or other hydrolysable group-terminated) silicone oils, or terminal hydrosilylated oils). For example, a particular triglyceride molecule can have three aliphatic fatty acid ester groups, at least one of which has at least one unsaturated carbon-carbon double bond. Mono- and di-glycerides also can be used when there is sufficient unsaturation in the fatty acid esters.

The unsaturated oil generally includes natural oils, for example any unsaturated vegetable or animal oils or fats; more specifically, the term “oil” generally refers to lipid structures (natural or synthetic), regardless of whether they are generally liquid at room temperature (i.e., oils) or solid at room temperature (i.e., fats). Examples of unsaturated oils include, but are not limited to, natural oils such as soybean oil (preferred), safflower oil, linseed oil, corn oil, sunflower oil, olive oil, canola oil, sesame oil, cottonseed oil, palm oil, poppy-seed oil, peanut oil, coconut oil, rapeseed oil, tung oil, castor oil, fish oil, whale oil, Abyssinian oil (preferred) or any mixture thereof.

Additionally, any partially hydrogenated vegetable oils or genetically modified vegetable oils can also be used. Examples of partially hydrogenated vegetable oils or genetically modified vegetable oils include, but are not limited to, high oleic safflower oil, high oleic soybean oil, high oleic peanut oil, high oleic sunflower oil and high erucic rapeseed oil (crambe oil). Alternatively or additionally, any unsaturated fatty acids (e.g., containing 10 to 24 carbons or 12 to 20 carbons in the unsaturated hydrocarbon chain) or esters thereof (e.g., alkyl esters, hydrocarbon esters containing from 1 to 12 carbon atoms), either individually or as mixtures, also can be used as an unsaturated oil according to the disclosure. The iodine values of the unsaturated oils preferably range from about 40 to 240 (e.g., about 80 to 240, about 120 to 160). When oils having lower iodine values are used, lower concentrations of hydrolysable silyl groups will be obtained in the silane-modified oil.

The unsaturated hydrolysable silane includes a silicon-based compound having an unsaturated hydrocarbon residue and at least one hydrolysable functional group bonded to a silicon atom. An example of a suitable unsaturated hydrolysable silane is represented by Formula I:

R″ₘSiR₄₋₍ₙ₊ₘ₎Xₙ [Formula I]

In Formula I, (i) X is a hydrolysable functional group, (ii) R is a terminal group or atom, (iii) R″ is an unsaturated hydrocarbon residue, and (iv) n is an integer ranging from 1 to 3, m is an integer ranging from 1 to 3, and n+m<=4. The value of n is preferably 2 or 3 (more preferably 3), thereby permitting more than one siloxane linkage in the crosslinked silane-modified oil and facilitating the formation of networked gel polymer. Generally, the unsaturated hydrolysable silane contains a single carbon-carbon unsaturation (i.e., m is 1) so that the silane is covalently bonded to the unsaturated oil without any undesired crosslinking between unsaturated oil molecules. In some aspects, however, the unsaturated hydrolysable silane is polyunsaturated (e.g., m is 2 or 3 and/or R″ is polyunsaturated). Preferred unsaturated hydrolysable silanes include vinyltrimethoxysilane, vinyltriethoxysilane, vinyltriacetoxysilane, allyidimethylacetoxysilane, allyltriisopropoxysilane, and allylphenyldiphenoxysilane. R″, R, and X can be chosen independently from of each other, and specific examples of the various groups are given below.

Examples of hydrolysable functional groups X include alkoxy (e.g., methoxy, ethoxy), carboxyloxy (e.g., acetoxy), or aryloxy groups. Optionally, X can be a halogen such as chloride or bromide, although the halogens are less preferred as they lead to formation of strong acids upon hydrolysis, which acids are preferably neutralized to prevent saponification of any fatty acid esters in the oil (e.g., triglyceride ester bonds). Thus, in some aspects, the hydrolysable functional groups (or hydrolysable silyl groups) do not include halogens. Most preferably, X is either a methoxy and/or acetoxy group. Such silanes are commonly available and their methods of manufacture are well known. Preferred are the silanes in which there are three hydrolysable groups present, such as vinyltrimethoxysilane or vinyltriacetoxysilane.

The terminal group R is preferably a hydrogen, a saturated hydrocarbon group, a saturated alicyclic hydrocarbon group, an aryl hydrocarbon group, a heterocyclic hydrocarbon group, or a combination thereof. The hydrocarbon groups generally containing from 1 to 30 carbon atoms (e.g., 1 to 10 carbon atoms, 1 to 6 carbon atoms). For example, R can be a hydrogen, a saturated alkyl hydrocarbon group, a substituted saturated alkyl hydrocarbon group, an aryl group, or a substituted aryl group. Alkyl groups can be any hydrocarbon including carbon atoms in either a linear or a branched configuration. Alkyl/aryl groups could be hydrocarbons or substituted hydrocarbons where the substitution includes heteroatoms, halogens, ethers, aldehydes, ketones, and the like. Preferred alkyl groups are methyl, ethyl, and fluoropropyl groups. In a preferred aspect, however, n is 3, m is 1, and the terminal group R is not present in the unsaturated hydrolysable silane.

The unsaturated hydrocarbon residue R″ preferably contains from 2 to 30 carbon atoms (e.g., 2 to 14 carbon atoms, 2 to 6 carbon atoms). Generally, unsaturated hydrocarbon residue R″ is monounsaturated; however, R″ can be polyunsaturated (e.g., a dienyl group). In an aspect, the unsaturated functionality of R″ is at a terminal end of R″ (i.e., R″ is CH₂═CH—R′— where R′ is a hydrocarbon residue containing from 0 to 12 carbon atoms) to facilitate the grafting of the unsaturated hydrolysable silane to the unsaturated oil. The hydrocarbon residues preferably include alkyl, substituted alkyl, aryl, or substituted aryl segments such as methyl, ethyl, propyl, and phenyl (e.g., CH₂—CH-ph-). Most preferably, R″ is either a vinyl (CH₂═CH—) or allyl (CH₂═CH—CH₂—) group.

### Silane-Modification of the Oils

Any suitable method can be used to make the silane-modified oil. In one aspect utilizing unsaturated oil, the relative amounts of the unsaturated oil and the unsaturated hydrolysable silane are adjusted according to the specific grafting reaction conditions (e.g., temperature, reaction time, free radical initiator). In some aspects, prior to the grafting reaction, the unsaturated hydrolysable silane is present in a molar excess relative to the unsaturated oil, for example with the molar ratio of the unsaturated hydrolysable silane to the unsaturated oil ranging from about 1 to about 20, about 2 to about 10, about 3 to about 8, or about 4 to about 6. For some applications it is desirable to have at least 1 mole of reactive silyl groups (i.e., the reactive, hydrolysable silane group covalently bonded to the unsaturated oil) per molecule of the unsaturated oil (e.g., fatty acid triglycerides) to ensure complete crosslink at or above the gel point. For other applications, less than 1 mole of reactive silyl groups per molecule of the unsaturated oil can be used where it is desirable for at least a portion of the unsaturated oil to not be crosslinked into the gel network.

Depending upon the desired application, the amount of uncrosslinked unsaturated oil left in the composition after crosslinking can be varied. If excess amounts of unsaturated hydrolysable silane are used, minimum amounts of uncrosslinked unsaturated oil will be left in the composition after crosslink (i.e., either (1) unsaturated oil molecules not containing a hydrolysable silyl group or (2) unsaturated oil molecules containing a hydrolysable silyl group that did not hydrolyze/condense to form a siloxane crosslink with another hydrolysable silyl group). If, however, relatively lower amounts of the unsaturated hydrolysable silane are used, a portion of the unsaturated oil will not be crosslinked into the gel network and will remain free, tending to leach/bleed from a crosslinked composition.

After the grafting reaction, all or at least a portion of the unsaturated oil molecules have at least one hydrolysable silyl group covalently bonded thereto via the unsaturated hydrocarbon chain, depending upon the desired end use application. In some aspects, substantially no uncrosslinked unsaturated oil is present in a crosslinked composition and/or able to leach from the crosslinked composition. For example, uncrosslinked/leachable oil can be from about 5 wt. % or less (e.g., about 2 wt. %, 1 wt. %, or 0.1 wt. % or less), relative to the initial amount of unsaturated oil. In many applications, such incomplete crosslink is undesirable and may lead to problems related to staining of areas surrounding the point(s) of application, poor performance and problems related to adhesion, water resistance, and/or aesthetic appearance. In others, such incomplete crosslink can be advantageous, for instance when the uncrosslinked unsaturated oil present in the crosslinked mixture is subjected to a subsequent process in order to further modify the mixture's properties and composition.

Free Radical Initiator

In one aspect, a free radical initiator assists in the grafting reaction of the unsaturated hydrolys able silane onto the unsaturated oil (e.g., via the unsaturated aliphatic chain of the unsaturated oil molecule). Any free radical initiator generally known in the art is appropriate, with thermal initiators that generate free radicals upon heating being preferred. Examples include, but are not limited to, organic peroxides, such as a benzoyl peroxide, di-t-butylperoxide, 2,5-dimethyl-2,5-di(t-butylperoxide)hexane, bis-(o-methylbenzoyl)peroxide, bis(m-methylbenzoyl)peroxide, bis(p-methylbenzoyl)peroxide, or similar monomethylbenzoyl peroxides, bis(2,4-dimethylbenzoyl)peroxide, or a similar dimethylbenzoyl peroxide, dicumylperoxide, t-butyl 3-isopropenylcumyl peroxide, butyl 4,4-bis(tert-butylperoxy)valerate, bis(2,4,6-trimethylbenzoyl) peroxide, or a similar trimethylbenzoyl peroxide.

The free radical initiator leads to higher portions of the reactive hydrolysable silyl group covalently bonded to the unsaturated oil and minimizes the risk of having an incomplete network upon crosslinking that permits free (i.e., non-crosslinked) unsaturated oil molecules to diffuse out of the bulk. Such diffusion of unreacted unsaturated oil molecules from the network has adverse effects on the physical properties of the gel network itself as well as the surrounding areas.

The initiator is added in any appropriate amount to ensure that the resulting composition will crosslink by grafting sufficient hydrolysable silyl groups onto the unsaturated oil. Preferably the initiator is used in an amount of about 0.1 wt. % to about 10 wt. % (e.g., about 0.2 wt. % to about 5 wt. % or about 0.5 wt. % to about 2 wt. %), relative to the weight of the unsaturated oil component.

Preferably, the free radical initiator is used in a reaction mixture that is either substantially free of or free of antioxidants and/or peroxide scavengers. In some cases, antioxidants and/or peroxide scavengers (e.g., t-butyl pyrocatechol, butylated hydroxy toluene, butylated hydroxy anisole, hydroquinone) are added to unsaturated silanes to prevent the spontaneous polymerization of the unsaturated silanes. However, the use of the free radical initiator without the antioxidant/peroxide scavenger promotes the silylation graft reaction while also reducing the rate of undesirable side reactions. Further, spontaneous polymerization of the unsaturated silanes was not observed in the various Example formulations prepared and analyzed.

Bonding

Any suitable bonding process can be used herein. For example, in one aspect, a suitable process for performing a graft reaction to form a water-curable, silane-modified oil includes preparing a reaction mixture that includes about 1 mole of unsaturated oil per 5 moles of the unsaturated hydrolysable silane and about 1 wt. % peroxide initiator (relative to the unsaturated oil) in a closed flask under an inert (e.g., nitrogen) atmosphere. The reaction mixture should be substantially water-free to prevent premature hydrolysis and/or siloxane crosslinking (e.g., sufficiently free of water to prevent reaction based time available for reaction, ambient temperature, pH, etc.). For example, the reaction mixture is pumped under a nitrogen blanket into a 2 L Parr reactor that has been purged with dry nitrogen for about 5 minutes to ensure dry atmosphere. The Parr reactor (from Parr Instrument Company, Moline, Ill., USA) is equipped with a mechanical stirrer, a sampling port and thermocouple well. The temperature of the reactor is then adjusted using an external controller and the mixture is heated while stirring at 200 rpm in order to mix the reactants and distribute the heat uniformly throughout the reactor.

Typical reaction temperatures are between about 100 deg. C. to about 350 deg. C. For common vinyl and unsaturated hydrolysable silanes, the reaction temperature is generally in the higher end of the range, (e.g., about 200 deg. C. to about 350 deg. C., or about 200 deg. C. to about 300 deg. C. When the unsaturated hydrocarbon residue R″ is an aryl residue (e.g., CH₂CH-ph-), however, lower reaction temperatures may be suitable (e.g., about 100 deg. C. to about 200 deg. C., or about 100 deg. C. to about 180 deg. C.). Since many of the unsaturated hydrolysable silanes have boiling points below the reaction temperature, care is taken to ensure that the reactor can withstand the pressure build-up during the reaction. At the end of the reaction, the heat is turned off, allowing the silane-modified oil to cool down to room temperature. Excess unreacted unsaturated hydrolysable silane can then be removed from the product by simple evaporation or be left in the product. The amount of reacted (i.e., covalently bonded) and unreacted hydrolysable silane in the oil is determined by placing a sample in a thermo-gravimetric analyzer (TGA) held at 160 deg. C. for a period of about 20-30 minutes. Any unreacted hydrolysable silane is volatilized away from the product, registering as a weight loss in the TGA. The concentration of the covalently bonded silane is calculated by subtracting the weight loss of the volatile fraction (i.e., unreacted silane) from the initial weight of unsaturated hydrolys able silane in the reaction mixture.

In another aspect, the silane-modified oil includes linear, branched, or cross-linked polymers comprising one or more silanol and/or hydrolysable siloxy residues. In particular, the polymeric materials comprise addition polymers produced from one or more ethylenically unsaturated monomers copolymerized with a monomer comprising a silanol or hydrolysable siloxy residue.

One group of suitable polymers includes those produced by polymerization of ethylenically unsaturated monomers using a suitable initiator or catalyst, such as those disclosed in U.S. Pat. No. 6,642,200. Suitable polymers may be selected from the group consisting of a synthetic polymer made by polymerizing one or more monomers selected from the group consisting of N,N-dialkylaminoalkyl acrylate, N,N-dialkylaminoalkyl methacrylate, N,N-dialkylaminoalkyl acrylamide, N,N-dialkylaminoalkylmethacrylamide, quaternized N,N dialkylaminoalkyl acrylate quaternized N,N-dialkylaminoalkyl methacrylate, quaternized N,N-dialkylaminoalkyl acrylamide, quaternized N,N-dialkylaminoalkylmethacrylamide, Methacryloamidopropyl-pentamethyl-1,3-propylene-2-ol-ammonium dichloride, N,N,N,N′,N′,N″,N″-heptamethyl-N″-3-(1-oxo-2-methyl-2-propenyl)aminopropyl-9-oxo-8-azo-decane-1,4,10-triammonium trichloride, vinylamine and its derivatives, allylamine and its derivatives, vinyl imidazole, quaternized vinyl imidazole and diallyl dialkyl ammonium chloride, N,N-dialkyl acrylamide, methacrylamide, N,N-dialkylmethacrylamide, C₁-C₁₂ alkyl acrylate, C₁-C₁₂ hydroxyalkyl acrylate, polyalkylene glyol acrylate, C₁-C₁₂ alkyl methacrylate, C₁-C₁₂ hydroxyalkyl methacrylate, polyalkylene glycol methacrylate, styrene, butadiene, isoprene, butane, isobutene, vinyl acetate, vinyl alcohol, vinyl formamide, vinyl acetamide, vinyl alkyl ether, vinyl pyridine, vinyl pyrrolidone, vinyl imidazole, vinyl caprolactam, acrylic acid, methacrylic acid, maleic acid, vinyl sulfonic acid, styrene sulfonic acid, acrylamidopropylmethane sulfonic acid (AMPS) and their salts. The polymer may optionally be branched or cross-linked by using branching and crosslinking monomers. Branching and crosslinking monomers include ethylene, glycoldiacrylate, divinylbenzene, and butadiene. Preferably the polymer comprises a synthetic polymer made by polymerizing isobutene with a molecular weight of less than 8,000, preferably between 500 and 8,000.

In one aspect, the monomer comprising a silanol or hydrolysable siloxy residue comprises the monomer of the following structure:

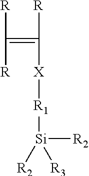

where each R is independently selected from the group consisting of hydrogen, C₁ to C₁₂ alkyl, and C₁ to C₁₂ substituted alkyl groups. Each X comprises a divalent alkylene radical comprising 2-12 carbon atoms. In one aspect each of the divalent alkylene radicals is independently selected from the group consisting of

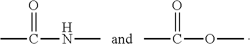

Each R₁ comprises a divalent alkylene radical comprising 2-12 carbon atoms. In one aspect each of the divalent alkylene radicals is independently selected from the group consisting of —(CH₂)ₛ— wherein s is an integer from 2 to 8 or from 2 to 4; —CH₂—CH(OH)—CH₂— and —CH₂—CH₂—CH(OH)—. Each R₂ is selected from OH, C₁-C₈ alkoxy and C₁-C₈ alkyl, and each R₃ is selected from OH and C₁-C₈ alkoxy. In one aspect R₃ is selected from OH and methoxy, ethoxy or propoxy groups

The Silane-Modified Oils

The silane-modified oil can have differing degrees of unsaturation depending upon the desired end use properties. Additionally, the silane-modified oil can have differing degrees of branching, aromaticity, molecular weight, chain length, functionalization with heteroatoms, or any other possible variation depending upon the desired end use properties.

As discussed above, the level of unsaturation can be modified either before, during, or after the grafting process. The silane-modified oil can have greater than or equal to zero double-bonds, or one or more double bonds, present in silane-modified oil. For example, if the silane-modified oil will be further modified by reactions needing the presence of double bonds, it can be advantageous for the silane-modified oil to contain an abundance of double bonds. In other aspects, the degree of unsaturation in the silane-modified oil is kept to a minimum, while in others the degree of unsaturation can be irrelevant depending upon the intended end-use application.

For instance, in one aspect the silane-modified oil has a degree of unsaturation that is substantially similar to that of the unsaturated oil. The similar degrees of unsaturation represent a minimization of undesirable coupling reactions between unsaturated oil carbon-carbon double bonds while promoting the grafting reaction of the unsaturated hydrolysable silane onto the unsaturated oil chains. The undesirable coupling reactions between unsaturated oil molecules (i.e., “bodying” reactions) tend to increase the molecular weight of the unsaturated oil while also reducing the available sites for unsaturated hydrolysable silane grafting. The reduction of available grafting sites further tends to result in bodied unsaturated oil molecules that, absent any hydrolysable silane functionality, will undesirably leach from a crosslinked composition.

The degree of unsaturation can be conveniently expressed by any of a variety of methods. For example, the total number of carbon-carbon double bonds in both the original unsaturated oil and the silane-modified oil product can be determined (e.g., by NMR spectroscopy) and compared. In some aspects, the unsaturated hydrocarbon chain can retain its carbon-carbon double bond, even though the position of the double bond changes as a result of the grafting reaction. Alternatively, the degree of unsaturation can be characterized by the iodine number (e.g., amount of iodine consumed by a substance, for example as determined by ASTM D1959, ASTM D5768, DIN 53241, or equivalent).

The relative retention of unsaturated character in the silane-modified oil product also can be expressed by its viscosity, which can remain similar or can be different than that of the reactant oil that was used, depending upon the desired end-use application. For example, when a low viscosity vegetable oil is employed as the unsaturated oil, the silane-modified oil product can have a similar low viscosity, which facilitates smooth, continuous film formation when deposited as a coating. In other applications, it can be desirable to adjust the viscosity either higher or lower depending upon the desired end use.

The silane-modified oil can be further characterized in terms of the particular structure of its hydrolysable silyl group(s), for example as expressed by Formula II:

—SiRₘR₃₋₍ₙ₊ₘ₎Xₙ [Formula II]

In Formula II, X and R can represent the same hydrolysable functional groups and terminal groups/atoms as in Formula I. In Formula II, n ranges from 1 to 3 (preferably 3), m ranges from 0 to 2, and n+m<=3. Because the hydrolysable silyl group of Formula II is covalently bonded to the unsaturated oil, R″ can represent both the unsaturated hydrocarbon residues of Formula I or the graft reaction product of the unsaturated hydrocarbon residues. As an example, R″ can represent the vinyl group (CH₂═CH—) or the ethylene graft reaction product of the vinyl group (—CH₂CH₂—), in the event that the unsaturated hydrolysable silane is polyunsaturated and/or covalently bonded to more than one unsaturated hydrocarbon chain. Generally, the hydrolysable silyl group is covalently bonded to the unsaturated hydrocarbon chain via a linking group R′″ that represents the graft reaction product of R. In this case, the hydrolysable silyl group that is directly covalently bonded to the unsaturated hydrocarbon chain (i.e., via the linking group R′″) can be represented by Formula IIa:

—R′″SiR″ₘR₃₋₍ₙ₊ₘ₎Xₙ [Formula IIa]

In one aspect, the silane-modified oil can be in the form of a particle. The particle comprises: (1) a particle core having an interfacial surface; (2) a silane-modified oil attached to said interfacial surface; and optionally (3) a polymer having a property. The silane-modified oil and optionally the polymer are attached to the interfacial surface of the particle core at different locations on the interfacial surface. In some aspects, the particle comprises two or more than two polymers and/or properties.

Particle Core

Any suitable particle core can be used, depending upon the desired attributes. In one aspect, the particle core is an inorganic particle, comprising hydroxyl functionality on the interfacial surface. In some instances, nanoparticles, either individually or as an agglomerate, are used as the particle core. As used herein, the term nanoparticle (either individually or as an aggregate) refers to a particle that is less than 500 nanometers in its longest dimension. In one aspect, the nanoparticles are from 1 to 500 nanometers, in another aspect from 150 to 250 nanometers, and in another aspect the nanoparticles are from 50 to 100 nanometers.

The desired benefit can guide the choice of the particle core to be used for any particular consumer product composition. For example, a particle (or agglomeration of particles), such as silicate particles (e.g., fumed silica), alumina silicates, metal oxides (e.g., zinc oxide, titanium dioxide), can be used as the particle core.

Other non-limiting examples of materials that can be used to form the particle core include colored and uncolored pigments, interference pigments, inorganic powders, and combinations thereof. These particulates can, for instance, be platelet shaped, spherical, elongated or needle-shaped, or irregularly shaped, surface coated or uncoated, porous or non-porous, charged or uncharged. Specific materials can include, but are not limited to, bismuth oxychloride, sericite, mica, mica treated with barium sulfate or other materials, zeolite, kaolin, silica, boron nitride, talc, aluminum oxide, barium sulfate, calcium carbonate, glass, and mixtures thereof.

Other pigments useful in the present invention can provide color primarily through selective absorption of specific wavelengths of visible light, and include inorganic pigments, organic pigments and combinations thereof. Examples of such useful inorganic pigments include iron oxides, ferric ammonium ferrocyanide, manganese violet, ultramarine blue, and Chrome oxide. Inorganic white or uncolored pigments useful in the present invention, for example TiO₂, ZnO, or ZrO₂, are commercially available from a number of sources. One example of a suitable particulate material contains the material available from U.S. Cosmetics (TRONOX TiO₂ series, SAT-T CR837, a rutile TiO₂). Particularly preferred are charged dispersions of titanium dioxide, as are disclosed in U.S. Pat. No. 5,997,887.

Particular colored or uncolored non-interference-type pigments have a primary average particle size of from 1 nm to 150,000 nm, alternatively from 10 nm to 5,000 nm, or from 20 nm to 1000 nm. Mixtures of the same or different pigment/powder having different particle sizes are also useful herein (e.g., incorporating a TiO₂ having a primary particle size of from about 100 nm to about 400 nm with a TiO₂ having a primary particle size of from about 10 nm to about 50 nm).

Interfacial Surface

The interfacial surface of the particle core can be either located directly on the surface of the particle core itself, or can be located one or more layers above the particle core if the particle core to be used is a coated particle core. When the particle core comprises a plurality of particles, the interfacial surface can extend over multiple particle surfaces.

Interfacial Surface Attachment

At least one silane-modified oil molecule, and optionally one or more polymers, are attached to the particle core's interfacial surface at different points. As used herein, “attached” can include any suitable means of attachment, such as bonding (e.g., covalent, ionic), or adsorption (e.g., van der Waals, Hydrogen bonding, etc.) depending upon the desired final properties of the consumer product composition.

In one aspect, a block co-polymer is used. Polymers having the same or contrasting properties can be incorporated into a single block co-polymer. The block co-polymer can be attached to the core at single or multiple points.

The polymer(s) have a chemical and/or physical property; optionally, at least one polymer's property contrasts with another polymer's property. A polymer's property can also or alternatively contrast with a property of the silane-modified oil. Examples of properties and corresponding contrasting properties can include, but are not limited to: hydrophobic and hydrophilic; acidic and basic; and anionic and cationic.

Contrasting properties of the polymer(s), either with the properties of other polymers or with the silane-modified oil, enable the resulting particle to adapt to its environment. For example, when there is a change in a parameter that affects a particular property, a first polymer's property will be expressed, and the first polymer's effect will be dominant over the second polymer's contrasting property. For example, a change in solvent polarity could trigger a conformational change in the polymer chains, resulting in a more hydrophobic or hydrophilic property being expressed. Other changes could include pH, water content, humidity, temperature, solvent content, electrolyte concentration, magnetic field, radiation exposure, etc. In a particular aspect, a polymer comprises not one but a plurality of properties such that it will be responsive to multiple stimuli (e.g., both solvent polarity and temperature.)

The inclusion of particles in a consumer product composition can thus lead to advantages such as, but not limited to, improved and uniform deposition of hydrophobic materials on surfaces of non-uniform surface energies. For example, the deposition of these hydrophobic materials onto the hair surface changes the surface energy. Furthermore, formulation of hydrophobic materials into an aqueous chassis (e.g., carrier) can be more easily accomplished. Conversely, the formulation of hydrophilic materials into a non-aqueous chassis can be more easily accomplished. In addition, the removal of the particles can be facilitated by changes in environment.

The selection of the polymer types, levels, and ratios depends on the product type, desired property, stimulus, and chassis used. In general, it is desirable to be able to deliver the particles in various chassis preserving their stability towards aggregation/flocculation and settling. For example, relatively large polymers may be selected to achieve entropic stabilization. In one aspect, the polymer has a molecular weight of greater than 500, in another aspect the molecular weight is more than 15,000. In a particular aspect, the polymer has a molecular weight from 1000 to 300,000. In aqueous chassis, the presence of ionic groups in a hydrophilic polymer will provide additional flocculation/aggregation stability.

In particular aspects, hydrophobic polymers can include, but are not limited to, fluorinated polystyrenes, polystyrenes, polyolefins (and functionalized, such as cyanides, halides, esters, pyrrolidone, carboxylic acids, carboxylic acid esters, hydroxyl, hydroxyl derivatives of carboxylic acid esters, amides, amines, glycidyl derivatives, etc.), polydienes, PDMS and functionalized PDMS, polybutylene oxides, polypropylene oxides, and alkyl derivatives and combinations thereof.

In particular aspects, hydrophilic polymers can include, but are not limited to, polyacrylates (and esters), other functionalized polyolefins, (such as PVA (polyvinyl alcohols and esters), PVA ethers, PVP (vinyl pyrrolidones), vinyl cyanides, phosphates, phosphonates, sulfates, sulfonates, etc.), polyethylenimine and other polyamines, polyethylene glycols and other polyethers, poly(styrene maleic anhydride), polyesters, polyureas, polyurethanes, polycarbonates, polyacrylamides, sugars and polymeric analogs, chitosan, and derivatives thereof and combinations thereof.

In order to have a robust responsive behavior (rapid and effective switching behavior upon a stimulus) conformational flexibility of the polymers is important. Therefore, a low glass transition temperature is desirable.

When the attachment mechanism is adsorption, the presence of multiple particle affinity groups on the polymer may be advantageous in order to achieve effective attachment under the appropriate conditions.

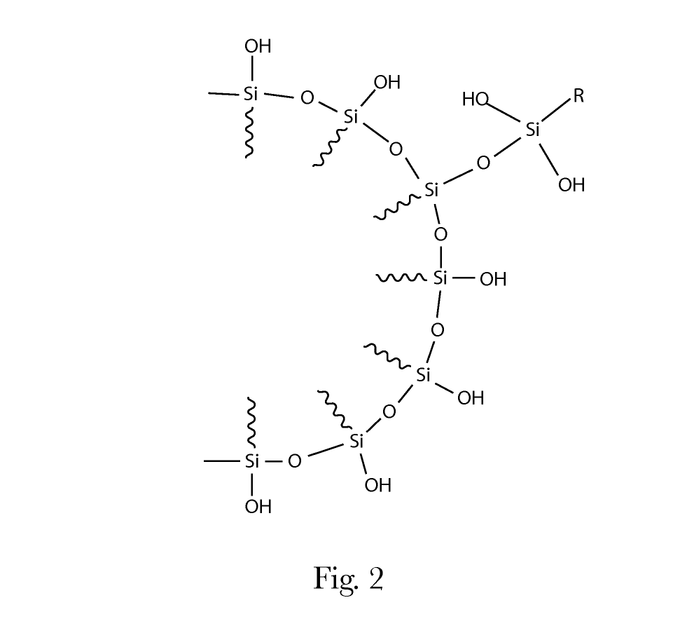

FIG. 2 illustrates generally a silane-modified oil bonded to the surface of a particle. An organo-functional silanol oil is shown attached to a silica surface.

Methods for Making Particles

In another aspect, the present invention provides methods for making particles for use in consumer product compositions. The method comprises: (1) providing a particle having an interfacial surface, (2) attaching a silane-modified oil (optionally having at least one property) to said interfacial surface; and optionally (3) attaching a polymer having a same or contrasting property or combinations thereof to said interfacial surface. Steps (2) and (3) can be performed in any appropriate order, including overlapping or simultaneously, depending on the particular polymers and methods of attachments desired. In aspects including a block copolymer, the first block can have a first property and the second block can have a second property; the properties can be either the same or contrasting or combinations thereof.

In general, the particles can be prepared/manufactured by using existing particulate raw materials as pre-formed particle cores (pigments, filler, etc.) and reacting functional groups on their surface with polymers or, adsorbing polymeric materials on their surface.

Alternatively, particles can be manufactured as the result of a polymerization reaction of soluble/emulsifiable monomers or macromonomers. The resulting polymer/co-polymer can form not only the solid core but also the attached polymers that provide the responsive feature. Additionally, the polymerization may be performed in the presence of particles (e.g. inorganic pigment) that can serve as an additional core material.

The creation of particles via polymerization reaction can provide a simple, fast, and economical process. For example, one can utilize aqueous emulsion polymerization of monomers containing at least one ethylene group in the presence of an initiator, a vinyl-terminated dimethylsiloxane macromonomer and, for instance, an alkene-containing polyethylenoxide. The silicone macromonomers can be emulsified into the aqueous medium with the other monomers using a surfactant in order to ascertain its participation to the polymerization reaction. After polymerization the resulting dispersion contains polymeric particles (latex) with attached macromonomers. Addition of inorganic particles (such as titanium dioxide, zinc oxide, silica, etc.) or other polymeric particles in the reaction mixture before the polymerization, also participate in the latex particles.

Typical emulsion polymerization monomers can include methyl methacrylate, acrylonitrile, ethyl acrylate, methacrylamide, styrene, etc. More hydrophilic monomers like acrylic acid and methacrylic acid may be copolymerized as well. Examples of PDMS macromonomers can include vinyl-terminated polydimethylsiloxanes, vinylmethylsiloxane-dimethylsiloxane copolymers, and methacroloxypropyl-terminated polydimethylsiloxanes. Examples of polar macromonomers can include polyoxyethylene esters of unsaturated fatty acid, polyoxyethylene ethers of fatty alcohols, vinyl-terminated polyethylenimine, and 2-(dimethylamino) ethyl methacrylate.

Similar results can be obtained when dispersion polymerization is attempted in an organic solvent instead of water. Typical solvents that can be used in this free radical dispersion polymerization include methylethyl ketone and isopropanol.

In the case where an inorganic particle (e.g., titanium dioxide, zinc oxide, or silica) is used in the aqueous reaction mixture, encapsulation of the particle with an unsaturated fatty acid polyoxyethylene ester or fatty alcohol polyoxyethylene ether followed by reaction with PDMS macromonomer can be another approach of creating similar responsive structures.

### Crosslinking and Gels

Depending upon the desired end-use application, the silane-modified oil can be cross-linked before, during, or after application to a substrate. For example, the silane-modified oil can be directly applied to surfaces, or it can further be processed to form a cross-lined gel network or a reactive particle before surface application.

Crosslinking of the silane-modified oils can be accomplished through reaction with the hydroxyl functional species, including either the inorganic hydroxyl functionalized particles, or the organic hydroxyl functionalized species, or both.

The silane-modified oil can be crosslinked by exposure to water, thereby hydrolyzing the hydrolysable silyl groups to silanol groups and subsequently condensing the silanol groups to form covalent intermolecular siloxane crosslinks in the silane-modified oil, or between the silane-modified oil and the hydroxyl functionalized species (e.g., the inorganic particle or the organic species, or both). In one aspect, the crosslinking water simply represents atmospheric moisture (e.g., up to about 5 vol. % water in air, about 0.5 vol. % to about 5 vol. %, about 1 vol. % to about 2 vol. %, alternatively about 20% to about 100% relative humidity). Thus, the composition comprising the silane-modified oil is simply applied to a substrate that is exposed to the atmosphere, and the silane-modified oil crosslinks gradually as the atmospheric moisture hydrolyzes the hydrolysable silyl groups. The rate of crosslink depends on the concentration of the hydrolysable silyl groups, the relative humidity, the temperature, and the layer thickness of the silane-modified oil applied to a substrate. The crosslinking temperature can be ambient temperature (e.g., about 25 deg. C.). Alternatively or additionally, the silane-modified oil can be maintained at or otherwise heated to a controlled temperature, for example up to about 80 deg. C. or about 25 deg. C. to about 60 deg. C. Further, pH can affect the crosslink rate. For instance, cross-linking can be facilitated by creating a more acidic environment where the silyl groups are more easily hydrolyzed to silanol groups, which are subsequently condensed to form crosslinks.

The rate of crosslink can further be accelerated using crosslinking catalysts known to accelerate moisture-induced reactions of hydrolysable silanes (generally known in the art as “accelerators”). Examples of suitable catalysts include titanium catalysts such as titanium naphthenate, tetrabutyltitanate, tetraisopropyltitanate, bis-(acetylacetonyl)-diisopropyltitanate, tetra-2-ethylhexyl-titanate, tetraphenyltitanate, triethanolam inetitanate, organosiloxytitanium compounds (such as those described in U.S. Pat. No. 3,294,739), and beta-dicarbonyl titanium compounds (such as those described in U.S. Pat. No. 3,334,067), both patents being herein incorporated by reference to show titanium catalysts. Alternatively, an organometallic tin condensation crosslink catalyst can be used to accelerate the rate of crosslink. Examples of tin carboxylate condensation crosslink catalysts include dibutyl tin dilaurate, dibutyl tin diacetate, dioctyl tin dilaurate, tin octoate, or mixtures thereof. Preferred catalysts include tetrabutyltitanate, tetraisopropyltitanate, and bis-(acetylacetonyl)-diisopropyltitanate. The amount of crosslinking catalyst preferably ranges from about 0.2 wt. % to about 6 wt. % (e.g., about 0.5 wt. % to about 3 wt. %) relative to the weight of the silane-modified oil. When present, the crosslinking catalyst is preferably provided as a mixture with the moisture-curable silane-modified oil so that the two components can be applied to a surface in a single operation.

In one aspect, the crosslinked silane-modified oil can be further characterized in terms of the particular structure of its covalent intermolecular siloxane crosslinks, for example as expressed by Formula III:

—R′″—Si(Y)₂—O—Si(Y)₂—R′″— [Formula III]

In Formula III, the Y moieties can independently represent —OH (i.e., a hydrolyzed but uncondensed silanol), —R, —R″, —O—Si(Y)₂—R′″—, and combinations thereof. The recursive definition of Y indicates that the siloxane crosslinks can be branched and need not be a 2-silicon crosslink. The R moieties can represent the same terminal groups/atoms as in Formula I, and the R″ moieties can represent the same unsaturated hydrocarbon residues and graft reaction products thereof as in Formula II. The R′″ moieties represent the same linking groups as in Formula II, thus generally representing a hydrocarbon residue having from 2 to 30 carbon atoms (e.g., 2 to 14 carbon atoms or 2 to 6 carbon atoms). Specifically, the R′″ moieties are the linking groups covalently bonded to the oil's unsaturated hydrocarbon chains at both ends of the intermolecular siloxane crosslinks, thus covalently linking at least two silane-modified oil molecules together. In an aspect of the crosslinked oil, (i) the unsaturated oil includes soybean oil; (ii) the Y moieties independently represent —OH, —O—Si(Y)₂—R′″—, and combinations thereof; and (iii) the R′″ moieties independently represent —CH₂CH₂—, —CH₂CH₂CH₂—, and combinations thereof.

In another aspect, the crosslinking of the silane-modified oil can be accomplished through bridging by the hydroxyl functionalized inorganic particles or the hydroxyl functionalized organic species, or both.

In the crosslinked silane-modified oil, substantially all of the oil molecules may be crosslinked to at least one other oil molecule via the intermolecular siloxane crosslinks. Additionally, the leaching of non-silylated oil molecules is limited. Once crosslinked, the silane-modified oil preferably has a gel content of at least about 70% (e.g., at least about 80%, at least about 90%, at least about 95%, or at least about 98%). The gel content of a crosslinked oil can be determined by equilibrating a sample of the crosslinked oil in a solvent (e.g., about 1 g to 2 g crosslinked oil per 50 ml of solvent, or 2 g crosslinked oil in 50 ml of solvent) for several hours. The solvent (along with any extracted/dissolved portion of the crosslinked oil) is then removed from the sample and dried to constant weight. The fraction of the crosslinked oil that is not extracted is the gel fraction. Suitable solvents include toluene and chloroform, although both give similar results. The gel fraction of an uncrosslinked silane-modified oil can be determined by first crosslinking the uncrosslinked sample according to a standard procedure. A sample of the uncrosslinked oil is combined with a crosslinking catalyst (e.g., about 5 g uncrosslinked oil with about 4 wt. % dibutyl tin dilaurate) is crosslinked in a closed chamber at a constant temperature and constant relative humidity for a fixed period (e.g., about 25 deg. C. and about 100% relative humidity for about 2 days). The crosslinked sample is extracted according to the foregoing procedure to determine the gel content.

Prior to use, the silane-modified oil is kept in a moisture-impervious packaging to maintain anhydrous conditions. In use, the composition can be brushed, sprayed, dipped, or otherwise applied onto a substrate by any common techniques using conventional equipment known in the art, and the resulting exposure to ambient moisture is sufficient to allow the composition to crosslink. The silane-modified oil also can be provided in a solution with a non-aqueous solvent or in a suspension with a non-aqueous solvent (e.g., alcohols such as ethanol, methanol, and the like), which solution or suspension can optionally include the crosslinking catalyst. The solution/suspension can then be sprayed onto a substrate to provide a thinner coating than might otherwise be possible with the concentrated silane-modified oil.

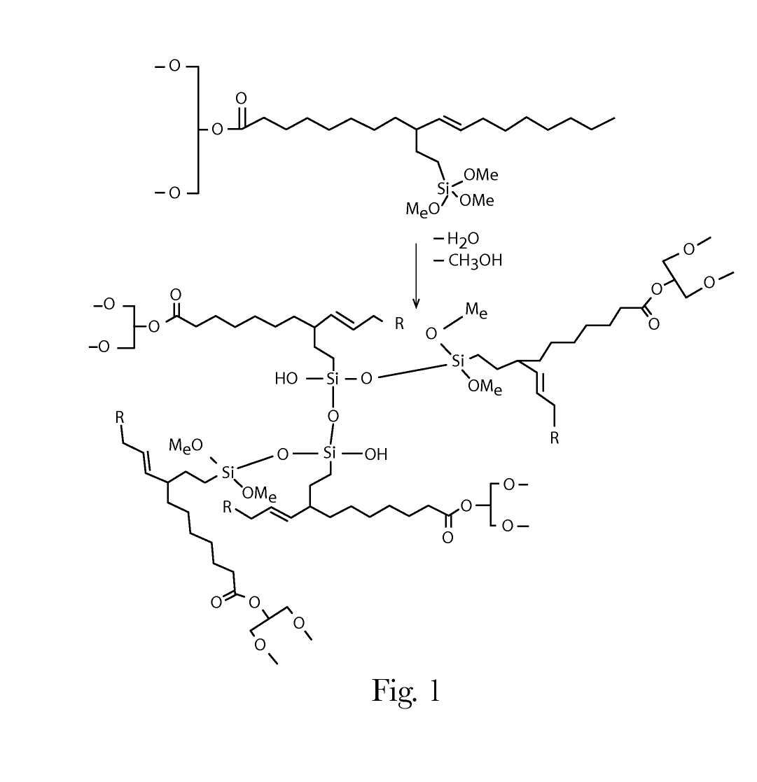

FIG. 1 illustrates the grafting and crosslinking processes and resulting compositions for a triglyceride unsaturated oil molecule having an 18-carbon unsaturated hydrocarbon chain (e.g., as a representative component of a fatty acid triglyceride) as one of the three fatty acid esters and vinyltrimethoxysilane. The grafting reaction (e.g., initiated by a peroxide free radical initiator, not shown) opens the vinyl group on the silane and grafts the silane to the hydrocarbon chain. The hydrolysable silane is covalently bonded to the aliphatic carbon chain at a position previously occupied by an olefinic carbon in the original oil. As a result of the grafting reaction, however, the carbon-carbon double bond migrates to an adjacent carbon-carbon pair. Thus, in the silane-modified oil, the hydrolysable silane is covalently bonded to the carbon chain at a position displaced by one carbon from the migrated carbon-carbon double bond. Crosslinking by exposure to water (e.g., atmospheric moisture) subsequently hydrolyzes the methoxy groups from the silicon, thereby forming silanol groups that can be further condensed with other silanol groups to form covalent intermolecular siloxane crosslinks in the crosslinked product.

The silylated oil may be stripped of any reagents used in making the oil prior to compounding into the consumer product. Said reagent-stripping may take for form of any known purification procedure known to one of ordinary skill in the art. For example, said reagent stripping may take the form of evaporative removal of any volatile reagents. Said evaporation may be performed under vacuum. The resulting purified silylated oil may be particularly useful for ease of formulation, stability and compatibility with home-use applications.

### Perfume and Perfume Microcapsules

The perfume component may comprise a component selected from the group consisting of perfume oils, mixtures of perfume oils, perfume microcapsules, pressure-activated perfume microcapsules, moisture-activated perfume microcapsules and mixtures thereof. Said perfume microcapsule compositions may comprise from 0.05% to 5%; or from 0.1% to 1% of an encapsulating material. In turn, the perfume core may comprise a perfume and optionally a diluent. Said perfume microcapsule may also be a particulate benefit agent.

Pressure-activated perfume microcapsules generally comprise core-shell configurations in which the core material further comprises a perfume oil or mixture of perfume oils. The shell material surrounding the core to form the microcapsule can be any suitable polymeric material which is impervious or substantially impervious to the materials in the core (generally a liquid core) and the materials which may come in contact with the outer substrate of the shell. In one aspect, the material making the shell of the microcapsule may comprise formaldehyde. Formaldehyde based resins such as melamine-formaldehyde or urea-formaldehyde resins are especially attractive for perfume encapsulation due to their wide availability and reasonable cost.

Moisture-activated perfume microcapsules, comprising a perfume carrier and an encapsulated perfume composition, wherein said perfume carrier may be selected from the group consisting of cyclodextrins, starch microcapsules, porous carrier microcapsules, and mixtures thereof; and wherein said encapsulated perfume composition may comprise low volatile perfume ingredients, high volatile perfume ingredients, and mixtures thereof;
(1) a pro-perfume;
(2) a low odor detection threshold perfume ingredients, wherein said low odor detection threshold perfume ingredients may comprise less than 25%, by weight of the total neat perfume composition; and
(3) mixtures thereof.

A suitable moisture-activated perfume carrier that may be useful in the disclosed multiple use fabric conditioning composition may comprise cyclodextrin. As used herein, the term “cyclodextrin” includes any of the known cyclodextrins such as unsubstituted cyclodextrins containing from six to twelve glucose units, especially beta-cyclodextrin, gamma-cyclodextrin, alpha-cyclodextrin, and/or derivatives thereof, and/or mixtures thereof. A more detailed description of suitable cyclodextrins is provided in U.S. Pat. No. 5,714,137. Suitable cylodextrins herein include beta-cyclodextrin, gamma-cyclodextrin, alpha-cyclodextrin, substituted beta-cyclodextrins, and mixtures thereof. In one aspect, the cyclodextrin may comprise beta-cyclodextrin. Perfume molecules are encapsulated into the cavity of the cyclodextrin molecules to form molecular microcapsules, commonly referred to as cyclodextrin/perfume complexes. The perfume loading in a cyclodextrin/perfume complex may comprise from 3% to 20%, or from 5% to 18%, or from 7% to 16%, by weight of the cyclodextrin/perfume complex.

The cyclodextrin/perfume complexes hold the encapsulated perfume molecules tightly, so that they can prevent perfume diffusion and/or perfume loss, and thus reducing the odor intensity of the multiple use fabric conditioning composition. However, the cyclodextrin/perfume complex can readily release some perfume molecules in the presence of moisture, thus providing a long lasting perfume benefit. Non-limiting examples of preparation methods are given in U.S. Pat. Nos. 5,552,378, and 5,348,667.

In one aspect, the hydroxyl functional organic species is relatively hydrophobic, preferably having a cLogP of from about 0.5 to about 14.5 (e.g. C4-C30), more preferably from about 2.9 to about 8.0 (e.g. C8-C18). The cLogP of the hydroxyl functional organic species is calculated using ChemBioDrawUltra 13.0 software.

### Optional Ingredients

### Hydroxyl Functional Organic Species

The hydroxyl functional organic species may be any organic species bearing at least one hydroxyl (—OH) moiety. Without being bound my theory it is believed that the hydroxyl functional organic species may participate in the cross-linking of the silane-modified oil through bridging by the hydroxyl moiety(ies) of the hydroxyl functional organic species

Non-limiting examples of hydroxyl functionalized organic species include monosaccharides, disaccharides, oligosaccharides and polysaccharides and functionalized monosaccharides, disaccharides, oligosaccharides and polysaccharides and their derivatives. Further non-limiting examples include cellulose, guar, starch, cyclodextrin, hydroxypropyl guar, hydroxypropyl cellulose, guar hydroxypropyltrimonium chloride, polyquarternium-10, dimethiconol, hydroxyl terminated polybutadiene, polyethylene oxide, polypropylene oxide, and poly(tetramethylene ether)glycol. In a particular aspect, the hydroxyl functionalized species comprises more than one hydroxyl group, preferably multiple hydroxyl groups, such that a bridge is formed between bonding sites on multiple silane-modified oils, thereby creating a gel. Said bridge may form as a result of nucleophilic attack of the hydroxyl-group of the hydroxyl functional organic species on the silyl-group of the silylated oil.

In one aspect, the hydroxyl functional organic species is an organo-silicone material such as a dimethiconol. The organo-silicone material may have a molecular weight of less than about 1,000,000 Daltons. The organo-silicone material may have a molecular weight of greater than about 1,000,000 Daltons. organo-silicone material may have a molecular weight of about 1,000,000 Daltons.

In one aspect, the hydroxyl functional organic species can be a polymer. In another aspect, the hydroxyl functional organic species comprises a vinyl polymer. In another aspect the hydroxyl functional organic species is a hydroxyl terminated polybutadiene.

In one aspect, the hydroxyl functional organic species is selected from the group consisting of glycols, poly-glycols, ethers, poly-ethers, polyalkylene oxides and derivatives thereof and mixtures thereof. In one aspect, the hydroxyl functional organic species is a polyethylene oxide, polypropylene oxide or a mixture thereof.

### Hydroxyl Functionalized Inorganic Particle

Hydroxyl functionalized inorganic particles are any inorganic solid particles comprising hydroxyl moieties on their surfaces and that are not dissolved in water or other solvents that may comprise a carrier for the compositions of the present invention. Non-limiting examples of suitable hydroxyl functionalized inorganic particles include metal oxides such as titania, alumina and metallocene, silica and zeolite.

As used herein, “silica” means particulate silicon dioxide. It would be appreciated by one of ordinary skill in the art that silica may take one of a number of forms including fumed silica, amorphous silica, precipitated silica, silica gel, and the like. It would be appreciated by one of ordinary skill in the art that particulate silica may include a plurality of surface-bound hydroxyl moieties (i.e. OH-groups).

In one aspect the hydroxyl functionalized inorganic particle may also be a particulate benefit agent. Non-limiting examples of hydroxyl functionalized inorganic particles that may also be particulate benefit agents include pigments, clays.

In one aspect, the hydroxyl functionalized inorganic particle may have an average particle size of from about 3 nm to about 500 um, preferably from about 3 nm to about 100 um, preferably from about 3 nm to about 50 um.

### Surfactants and Emulsifiers

The compositions of the present invention may comprise one or more surfactants or emulsifiers. The surfactant or emulsifier component is included in personal care compositions of the present invention to provide cleansing performance. The surfactant may be selected from anionic surfactant, zwitterionic or amphoteric surfactant, or a combination thereof. Suitable surfactant components for use in the composition herein include those which are known for use in hair care, fabric care, surface care or other personal care and/or home care cleansing compositions.

Suitable nonionic surfactants include, but not limited to, aliphatic, primary or secondary linear or branched chain alcohols or phenols with alkylene oxides, generally ethylene oxide and generally 6-30 ethylene oxide groups. Other suitable nonionic surfactants include mono- or di-alkyl alkanolamides, alkyl polyglucosides, and polyhydroxy fatty acid amides.

Non-limiting examples of suitable anionic surfactants are the sodium, ammonium, and mono-, di-, and tri-ethanolamine salts of alkyl sulfates, alkyl ether sulfates, alkaryl sulfonates, alkyl succinates, alkyl sulfosuccinate, N-alkoyl sarcosinates, alkyl phosphates, alkyl ether phosphates, alkyl ether carboxylates, and alpha-olefin sulfonates. The alkyl groups generally contain from 8 to 18 carbon atoms and may be unsaturated. The alkyl ether sulfates, alkyl ether phosphates, and alkyl ether carboxylates may contain from 1 to 10 ethylene oxide or propylene oxide units per molecule, and preferably contain 2 to 3 ethylene oxide units per molecule. Examples of anionic surfactants include sodium or ammonium lauryl sulfate and sodium or ammonium lauryl ether sulfate. Suitable anionic surfactants useful in the current invention are generally used in a range from 5% to 50%, preferably from 8% to 30%, more preferably from 10% to 25%, even more preferably from 12% to 22%, by weight of the composition.

Nonlimiting examples of suitable cationic surfactants include water-soluble or water-dispersible or water-insoluble compounds containing at least one amine group which is preferably a quaternary amine group, and at least one hydrocarbon group which is preferably a long-chain hydrocarbon group. The hydrocarbon group may be hydroxylated and/or alkoxylated and may comprise ester- and/or amido- and/or aromatic-groups. The hydrocarbon group may be fully saturated or unsaturated.

In one aspect, the level of surfactant may range from 0.5% to 95%, or from 2% to 90%, or from 3% to 90% by weight of the consumer product compositions.

Suitable zwitterionic or amphoteric surfactants for use in the composition herein include those which are known for use in hair care or other personal cleansing compositions. Concentration of such amphoteric surfactants preferably ranges from 0.5% to 20%, preferably from 1% to 10%. Non-limiting examples of suitable zwitterionic or amphoteric surfactants are described in U.S. Pat. Nos. 5,104,646 and 5,106,609, both to Bolich, Jr. et al.

The amphoteric surfactants suitable for use in the present invention can include alkyl amine oxides, alkyl betaines, alkyl amidopropyl betaines, alkyl sulfobetaines, alkyl glycinates, alkyl carboxyglycinates, alkyl amphopropionates, alkyl amidopropyl hydroxysultaines, acyl taurates, and acyl glutamates wherein the alkyl and acyl groups have from 8 to 18 carbon atoms.

Non-limiting examples of other anionic, zwitterionic, amphoteric, cationic, nonionic, or optional additional surfactants suitable for use in the compositions are described in McCutcheon's, Emulsifiers and Detergents, 1989 Annual, published by M. C. Publishing Co., and U.S. Pat. Nos. 3,929,678; 2,658,072; 2,438,091; and 2,528,378.

### Preservative

Preservatives may be useful in the present invention to ensure long-term stability of the product on-shelf relative to oxidation, microbial insult and other potential undesirable chemical transformations. Non-limiting examples of preservatives include anti-microbial preservatives and anti-oxidants.

Preferred anti-microbial preservatives include but are not limited to Benzalkonium chloride, Benzethonium chloride, Benzoic Acid and salts, Benzyl alcohol, Boric Acid and salts, Cetylpyridinium chloride, Cetyltrimethyl ammonium bromide, Chlorobutanol, Chlorocresol, Chorhexidine gluconate or Chlorhexidine acetate, Cresol, Ethanol, Hydantoins, Imidazolidinyl urea, Metacresol, Methylparaben, Nitromersol, o-Phenyl phenol, Parabens, Phenol, Phenylmercuric acetate/nitrate, Propylparaben, Sodium benzoate, Sorbic acids and salts, B-Phenylethyl alcohol, Thimerosal, and combinations thereof.

A preferred class of preservative as antioxidants. Antioxidants are added to minimize or retard oxidative processes that occur upon exposure to oxygen or in the presence of free radicals.

Preferred antioxidant preservatives include but are not limited to a-tocopherol acetate, Acetone sodium bisulfite, Acetylcysteine, Ascorbic acid, Ascorbyl palmitate, Butylated hydroxyanisole (BHA), Butylated hydroxytoluene (BHT), Citric acid, Cysteine, Cysteine hydrochloride, d-a-tocopherol natural, d-a-tocopherol synthetic, Dithiothreitol, Monothioglycerol, Nordihydroguaiaretic acid, Propyl gallate, Sodium bisulfite, Sodium formaldehyde sulfoxylate, Sodium metabisulfite, Sodium sulfite, Sodium thiosulfate, Thiourea, Tocopherols, and combinations thereof.

### Particulate Benefit Agents

Particulate benefit agents are solid particles that are not dissolved in water or other solvents that may comprise a carrier for the compositions of the present invention and that impart a benefit in use. Non-limiting examples of particulate benefit agents include pigments, clays, personal care actives such as anti-dandruff actives and anti-perspirant actives and encapsulated liquid actives including perfume microcapsules.

The particulate benefit agent may be of any size appropriate to the use and benefit to be derived. In one aspect, the particulate benefit agent has an average particle size of less than about 500 microns. In another aspect, the particulate benefit agent has an average particle size of less than about 100 microns. In another aspect, the particulate benefit agent has an average particle size of greater than about 3 nm. In another aspect, the particulate benefit agent has an average particle size of from about 1 micron to about 50 microns.

The particulate benefit agent may be platelet shaped, spherical, elongated or needle-shaped, or irregularly shaped, surface coated or uncoated, porous or non-porous, charged or uncharged or partially charged with either a positive charge or a negative charge. The particulate benefit agent may be added to the compositions as a powder or as a pre-dispersion.

Pigments include colored and uncolored pigments, interference pigments, optical brightener particles, and mixtures thereof. The average size of such particulates may be from about 0.1 microns to about 100 microns. These particulate materials can be derived from natural and/or synthetic sources.

Suitable organic powders particulate benefit agents include, but are not limited, to spherical polymeric particles chosen from the methylsilsesquioxane resin microspheres, for example, Tospearl™ 145A, (Toshiba Silicone); microspheres of polymethylmethacrylates, for example, Micropearl™ M 100 (Seppic); the spherical particles of crosslinked polydimethylsiloxanes, for example, Trefil™ E 506C or Trefil™ E 505C (Dow Corning Toray Silicone); spherical particles of polyamide, for example, nylon-12, and Orgasol™ 2002D Nat C05 (Atochem); polystyrene microspheres, for example Dyno Particles, sold under the name Dynospheres™, and ethylene acrylate copolymer, sold under the name FloBead™ EA209 (Kobo); aluminium starch octenylsuccinate, for example Dry Flo™ (National Starch); microspheres of polyethylene, for example Microthene™ FN510-00 (Equistar), silicone resin, polymethylsilsesquioxane silicone polymer, platelet shaped powder made from L-lauroyl lysine, and mixtures thereof.

Also useful herein are interference pigments. Herein, “interference pigments” means thin, platelike layered particles having two or more layers of controlled thickness. The layers have different refractive indices that yield a characteristic reflected color from the interference of typically two, but occasionally more, light reflections, from different layers of the platelike particle. The most common examples of interference pigments are micas layered with about 50-300 nm films of TiO₂, Fe₂O₃, silica, tin oxide, and/or Cr₂O₃. Such pigments often are pearlescent. Pearlescent pigments reflect, refract and transmit light because of the transparency of pigment particles and the large difference in the refractive index of mica platelets and, for example, the titanium dioxide coating. Intereference pigments are available commercially from a wide variety of suppliers, for example, Rona (Timiron™ and Dichrona™), Presperse (Flonac™), Englehard (Duochrome™), Kobo (SK-45-R and SK-45-G), BASF (Sicopearls™) and Eckart (Prestige™). In one aspect, the average diameter of the longest side of the individual particles of interference pigments is less than about 75 microns, and alternatively less than about 50 microns.

Other pigments useful in the present invention can provide color primarily through selective absorption of specific wavelengths of visible light, and include inorganic pigments, organic pigments and combinations thereof. Examples of such useful inorganic pigments include iron oxides, ferric ammonium ferrocyanide, manganese violet, ultramarine blue, and chromium oxide. Organic pigments can include natural colorants and synthetic monomeric and polymeric colorants. An example is phthalocyanine blue and green pigment. Also useful are lakes, primary FD&C or D&C lakes and blends thereof. Also useful are encapsulated soluble or insoluble dyes and other colorants. Inorganic white or uncolored pigments useful in the present invention, for example TiO₂, ZnO, or ZrO₂, are commercially available from a number of sources, for example, TRONOX TiO₂ series, SAT-T CR837, a rutile TiO2 (U.S. Cosmetics). Also suitable are charged dispersions of titanium dioxide, disclosed in U.S. Pat. No. 5,997,887, issued to Ha et al.

Clays include silicate and aluminosilicate minerals with layered structures. Non-limiting examples of clays include the smectite group clay minerals such as bentonite, montmorillonite, beidellite, nontronite, saponite, hectorite, sauconite, stevensite, and the like; vermiculite group clay minerals such as vermiculite, and the like; kaolin minerals such as halloysite, kaolinite, endellite, dicite, and the like; phyllosilicates such as talc, pyrophyllite, mica, margarite, muscovite, phlogopite, tetrasilicic mica, taeniolite, and the like; serpentine group minerals such as antigorite and the like; chlorite group minerals such as chlorite, cookeite, nimite, and the like. These layered inorganic compounds can be of natural products or of synthetic products. These can be singly used or used in combination of two or more.

Anti-dandruff actives are actives which, when deposited on the scalp, mitigate the formation of dandruff. The anti-dandruff active may be selected from the group consisting of: pyridinethione salts; azoles, such as ketoconazole, econazole, and elubiol; selenium sulphide; particulate sulfur; keratolytic agents such as salicylic acid; and mixtures thereof. Pyridinethione salts may be suitable anti-dandruff active particulates. In an aspect, the anti-dandruff active may be a 1-hydroxy-2-pyridinethione salt and is in particulate form. In an aspect, the concentration of pyridinethione anti-dandruff particulate ranges from about 0.01 wt % to about 5 wt %, or from about 0.1 wt % to about 3 wt %, or from about 0.1 wt % to about 2 wt %. In an aspect, the pyridinethione salts are those formed from heavy metals such as zinc, tin, cadmium, magnesium, aluminium and zirconium, generally zinc, typically the zinc salt of 1-hydroxy-2-pyridinethione (known as “zinc pyridinethione” or “ZPT”), commonly 1-hydroxy-2-pyridinethione salts in platelet particle form. In an aspect, the 1-hydroxy-2-pyridinethione salts in platelet particle form have an average particle size of up to about 20 microns, or up to about 5 microns, or up to about 2.5 microns. Salts formed from other cations, such as sodium, may also be suitable.

Anti-perspirant actives include any compound, composition or other material having antiperspirant activity. More specifically, the antiperspirant actives may include astringent metallic salts, especially inorganic and organic salts of aluminum, zirconium and zinc, as well as mixtures thereof. Even more specifically, the antiperspirant actives may include aluminum-containing and/or zirconium-containing salts or materials, such as, for example, aluminum halides, aluminum chlorohydrate, aluminum hydroxyhalides, zirconyl oxyhalides, zirconyl hydroxyhalides, and mixtures thereof.

### Other

Depending on the form of consumer product in which they are used (e.g., shampoo, liquid soap, bodywash, laundry detergent, fabric softener), these compositions may further contain ingredients selected from fatty alcohols having 8 to 22 carbon atoms, opacifiers or pearlescers such as ethylene glycol esters of fatty acids (e.g., ethylene glycol distearate), viscosity modifiers, buffering or pH adjusting chemicals, water-soluble polymers including cross-linked and non cross-linked polymers, foam boosters, dyes, coloring agents or pigments, herb extracts, hydrotopes, enzymes, bleaches, fabric conditioners, optical brighteners, stabilizers, dispersants, soil release agents, anti-wrinkle agents, chelants, anti corrosion agents, and mixtures thereof.

### EXAMPLES

The following are non-limiting examples of the present invention. The examples are given solely for the purpose of illustration and are not to be construed as limitations of the present invention, as many variations thereof are possible without departing from the spirit and scope of the invention, which would be recognized by one of ordinary skill in the art.

In the examples, all concentrations are listed as weight percent, unless otherwise specified and may exclude minor materials such as diluents, filler, and so forth. The listed formulations, therefore, comprise the listed components and any minor materials associated with such components. As is apparent to one of ordinary skill in the art, the selection of these minors will vary depending on the physical and chemical characteristics of the particular ingredients selected to make the present invention as described herein.

### Examples

### Material Synthesis

### Example 1

### Silylation, Option 1

Soybean oil (290 g), vinyltrimethoxysilane (246 g) and 2,5-bis(tert-butylperoxy)-2,5-dimethylhexane peroxide (LUPEROX 101) initiator (2.90 g) were mixed in a closed flask. The mixture was pumped using a nitrogen blanket into a 2 L Parr hydrogenator (from Parr Instrument Company, Moline, Ill., USA) that was purged with nitrogen for 5 minutes prior to the introduction of the reaction mixture to ensure an anhydrous atmosphere. The temperature of the reactor was set to 240 deg. C. and the agitation was kept at 200 rpm in order to mix the reactants and distribute heat uniformly in the system. The silylated soybean oil reaction product after 10 hours of reaction time was collected.

### Example 2

### Silylation, Option 2

In a 2 L Parr 4520 high pressure reactor equipped with overhead stir motor and thermocouple temperature control was placed soy oil (290 g), vinyltrimethoxysilane (246 g) and Luperox 101 (2,5 bis-(tert-butyl peroxy)-2,5-dimethylhexanediperoxide, 2.90 g) initiator. The reaction was heated at 225° C. for 24 h, and then cooled to RT.

On average, silylated soybean oils were synthesized using 1:1, 2:1 and 3:1 molar ratios of VTMOS to soybean oil. These yielded an average degree of silylation of the oil of 0.7, 1.5 and 2.4 moles of silyl-groups per mole of oil, respectively.

On average, silylated Abyssinian oils synthesized using 1:1 and 2:1 a ratios of VTMOS to Abyssinian oil yielded an average degree of silylation of the oil of 0.8 and 1.3 moles of silyl-groups per mole of oil, respectively.

On average, silylated high-oleic soybean oil synthesized using 1:1 and 2:1 a ratios of VTMOS to high-oleic soybean oil yielded an average degree of silylation of the oil of 0.8 and 1.7 moles of silyl-groups per mole of oil, respectively.

On average, silylated canola oil synthesized using 1:1 and 2:1 ratios of VTMOS to canola oil yielded an average degree of silylation of the oil of 0.9 and 1.4 moles of silyl-groups per mole of oil, respectively.

All silylated oils were assayed for silyl-content by Thermogravimetric analysis after purification as outlined in Example 3.

### Example 3

### Removal of Excess Reagent from Silylation Reactions

Excess silylating reagent was removed by placing crude reaction product on a rotary evaporator and stripping under vacuum (0.1-10 mmHg) at approximately 80° C. for 3-5 hrs.

### Example 4

Dibutyl tin dilaurate (0.1 g) was added to the sample in Example 1 (5 g). The resulting sample can be used directly or can be heated to a temperature up to 100° C. in the presence of humidity (ambient to 100% RH) before further use. (“RH”=relative humidity)

### Example 5

### Soy-Si-Particle

The silylated soy from Example 1 (5 g) was mixed with 0.10, 0.20 and 0.55 g of a particle size ranging from 0.003-500 um. The resulting sample can be used directly or can be heated to a temperature up to 100° C. in the presence of humidity (ambient to 100% RH).

Using an appropriately functionalized hydroxylated particle of similar size and the above procedure of this Example, the following modified soy particles could be accessed to one with ordinary skill in the art:

| | | | |
|---|---|---|---|
| -soy-Si-alumina | -soy-Si-metal oxide | -soy-Si-zeolite | ---soy-Si—OH- |
| | | | resin |
| -soy-Si-cellulose | -soy-Si-cyclodextrin | -soy-Si- | -soy-Si-starch |
| | | metallocene | |
| -soy-Si-silica | | | |

### Example 6

### Soy-Si-Polymer

To the silylated soy from Example 1 (5 g) is added dimethiconol (5 g). The resulting mixture can be used directly or can be heated to a temperature up to 100° C. in the presence of humidity (ambient to 100% RH). The resulting product can then be formulated accordingly, as in the consumer product examples below.

Using the appropriately functionalized polymer and the procedure from Example 5, a variety of soy-derived particle interpenetrating networks can be made, including:

| | | |
|---|---|---|
| -soy-Si-PEO | -soy-Si-PPO | -soy-Si-PTMG |

-soy-Si-hydroxyl terminated Polybutadiene

### Example 7

The silylated soy from Example 1 (5 g) was mixed with dimethiconol (5 g) and 0.10, 0.20 or 0.55 g of a hydroxyl functionalized particle having a particle size ranging from 0.003-500 um. The resulting sample can be used directly or can be heated to a temperature up to 100° C. in the presence of humidity (ambient to 100% RH). The resulting product is then formulated accordingly, such as in the consumer product examples herein.

### Examples

### Emulsions

All compositions evaluated for intrinsic performance may be prepared as aqueous emulsions per Examples 8-10, below. Silylated oils were prepared as above and emulsified using sodium dodecyl sulfate (typically at 30% oil to 0.75% SDS) using standard emulsification procedures. Compositions were prepared using an emulsified silylated oil and optionally a hydroxylated organic species or hydroxylated inorganic particle.

Hydroxy terminated PDMS (dimethiconol) was used as received as a prepared emulsion. Two samples were commercially prepared (DC1872, a 68000 cSt dimethiconol from Dow Corning, or MEM 1788 from Xiameter, a 2000000 cSt dimethiconol). An intermediate molecular weight (1000000 cSt dimethiconol) was prepared by emulsion polymerization of silanol-terminated dimethylsiloxane oligomers with dodecylbenzene sulfonic acid.

The resulting materials (e.g. silylated oil, silylated oil+catalyst, silylated oil+silica, silylated oil+dimethiconol, or silylated oil+silica+dimethiconol) in Examples 1-7 can also be made into a simple emulsion of at least 0.1% test material concentration (wt/wt), in deionized water, with a particle size distribution which is stable for at least 48 hrs at room temperature. Those skilled in the art will understand that such emulsions can be produced using a variety of different surfactants or solvents, depending upon the characteristics of each specific material. Examples of surfactants & solvents which may be successfully used to create such suspensions include: ethanol, Isofol®, Arquad® HTL8-MS or 2HT-75, Glycerol monooleate, Tergitol™ 15-S, Tergitol™ TMN, Tergitol NP, Tween, Span, linear alkyl sulfates such as sodium dodecyl sulfate, or Brij and mixtures thereof. Those skilled in the art will understand that such suspensions can be made by mixing the components together using a variety of high shear mixers. Examples of suitable homogenizers include an IKA® Ultra-Turrax or Silverson.

### Example 8

The silylated oil from Example 1 can also be made into a simple emulsion of at least 0.1% test material concentration (wt/wt), in deionized water, with a particle size distribution which is stable for at least 48 hrs at room temperature. The emulsion can be prepared using solvents, surfactants, and processing equipment as described above.

### Example 9

The emulsified silylated oil from Example 8 is mixed with fused silica having a particle size ranging from 0.003-5 um or other hydroxyl functionalized particle of similar particle size in ratios of 1:0.01 to 1:10.

### Example 10

The emulsified silylated oil from Example 8 is mixed with a fused silica having a particle size ranging from 0.003-5 um (or other hydroxyl functionalized particle) in ratios of 1:0.01 to 1:10 and with an emulsified hydroxyfunctional polymer, such as dimethiconol.

### Examples

### Intrinsic Performance

Examples demonstrating the intrinsic performance of composition of the present invention are depicted in Tables 1-5. The silane-modified oils used in the examples in tables 1-5 may be purified as per example 3 prior to compounding.

Fabric substrates were treated with emulsion compositions as indicated in the tables to yield 1 mg, 3 mg or 10 mg of total oil with oil being silylated oil, OH-functional polymer, or silylated oil+OH-functional polymer) per gram of fabric. All treated substrates were dried and allowed to equilibrate for at least 24 hours before testing. Fabrics used in the secant modulus testing were 100% Mercerized Combed Cotton Warp Sateen Fabric, approximately 155 grams/square meter, Style #479 available from Test Fabrics, West Pittston Pa. Fabrics used in the Time to Wick measurements were type CW120 stripped, no Brightener available from EMC. Compositions depicted in Table 3 were further pH-adjusted prior to use to a pH of 10.5 using 1M NaOH solution.

Hair substrates used in the testing were medium brown, not special quality hair switches, available from International Hair Importers & Products, Glendale, N.Y. Hair substrates were treated with emulsion compositions as indicated in the tables to yield 10 mg of total oil (with oil being silylated oil, OH-functional polymer, or silylated oil+OH-functional polymer) per gram of substrate and dried at 70F/50% RH (relative humidity) followed by 15 minutes in a 50 C oven 24 hours later.

### Time-to Wick

Time to wick is a measure of the compostions' capacity to impart repellency to a treated fabric. Without being bound by theory an increased Time to Wick is believed to correlate with an increase the a fabric repellency relative to staining. The fabric Time to Wick property is measured as follows.

The test is conducted in a room or chamber with air temperature of 20 to 25° C. and Relative Humidity of 45-55%. All fabrics and paper products used in the test are equilibrated in the temperature and humidity condition of the test location for at least 24 hrs prior to collecting measurements. The treated test fabric is cut into 10 squares, each approximately 1.25″×1″ in size. On a flat, horizontal and level, impermeable surface, place 10 individual squares, on top of a single sheet of kitchen paper towel (e.g. Bounty). The surface facing upwards, which is not in contact with the paper towel, is the surface that was placed in direct contact with the treatment composition during fabric preparation. Visually confirm that the fabric is lying flat and in uniform contact with the paper towel before proceeding.

The flat-lying fabric is then tested for the Time to Wick measurement. Distilled Water is used as the testing liquid. Automated single or multi-channel pipettes (e.g. Rainin, Gilson, Eppendorf), are used to deliver a liquid droplet size of 300 mL of the testing liquid onto the fabric surface. A stop-watch or timer is used to count time in minutes and seconds, from the moment when the liquid droplet contacts the fabric surface. The timer is stopped when the whole droplet of the test liquid is absorbed into the fabric. The time-point when the liquid droplet wets into the fabric is determined by visual observation. The time period shown elapsed on the timer is the Time to Wick Measurement. The test is stopped after 60 minutes if wetting of the liquid droplet has not been seen yet, and the Time to Wick measurement is recorded as >60 minutes in this case. If wetting of the liquid is seen immediately upon contact of the droplet with the fabric surface, then the Time to Wick property is recorded as 0 for that fabric. A total of 10 droplets are measured at different point on the test fabric and these 10 measurements are averaged to provide the reported Time to Wick value.

### Reduction in Secant Modulus

Reduction in Secant Modulus (RSM) is a measure of the compostions' capacity to impart softness to a treated fabric. Without being bound by theory it is believed that a lower secant modulus correlates with a more flexible fabric which will be perceived as softer by consumers. Note that RSM is reported as a reduction in secant modulus versus a control, so that a higher reported value correlates with a lower secant modulus and a superior softness result.

The RSM measurement is performed using a commercial tensile tester with computer interface for controlling the test speed and other test parameters, and for collecting, calculating and reporting the data. RSM testing was run using an Instron 5544 Testing System running the Bluehill software package. The test is conducted in a room or chamber with air temperature controlled to 20-25° C. and Relative Humidity (RH) controlled to 50%. All fabrics used in the test are equilibrated in the temperature and humidity condition of the test location for at least 16 hrs prior to collecting measurements.

During testing, the load cell is chosen so that the tensile response from the sample tested will be between 10% and 90% of the capacity of the load cells or the load range used. Typically a 500N load cell is used. The grips are selected such that they are wide enough to fit the fabric specimen and minimize fabric slippage during the test. Typically pneumatic grips set to 60 psi pressure and fitted with 25.4 mm-square crosshatched faces are used. The instrument is calibrated according to the manufacturer's instructions. The grip faces are aligned and the gauge length is set to 25.4 mm (or 1 inch). The fabric specimen is loaded into the pneumatic grips such that the warp direction is parallel to the direction of crosshead motion. Sufficient tension is applied to the fabric strip to eliminate observable slack, but such that the load cell reading does not exceed 0.5N. The specimens are tested with a multi-step protocol as follows:
(Step 1) Go to a strain of 10% at a constant rate of 50 mm/min and then return to 0% strain at a constant rate of 50 mm/min. This is the first hysteresis cycle.
(Step 2) Hold at 0% strain for 15 seconds and re-clamp the specimen to eliminate any observable slack and maintain a 25.4 mm gauge length without letting the load cell reading exceed 0.5N
(Step 3) Go to a strain of 10% at a constant rate of 50 mm/min and then return to 0% strain at a constant rate of 50 mm/min. This is the second hysteresis cycle.
(Step 4) Hold at 0% strain for 15 seconds and re-clamp the sample to eliminate any observable slack and maintain a 25.4 mm gauge length without letting the load cell reading exceed 0.5N
(Step 5) Go to a strain of 10% at a constant rate of 50 mm/min and then return to 0% strain at a constant rate of 50 mm/min. This is the third hysteresis cycle.
(Step 6) Hold at 0% strain for 15 seconds and re-clamp the sample to eliminate any observable slack and maintain a 25.4 mm gauge length without letting the load cell reading exceed 0.5N
(Step 7) Go to a strain of 10% at a constant rate of 50 mm/min and then return to 0% strain at a constant rate of 50 mm/min. This is the fourth hysteresis cycle.

The resulting tensile force-displacement data from the fourth hysteresis cycle (step 7) are converted to stress-strain curves using the initial sample dimensions, from which the secant modulus used herein, is derived. The initial sample dimensions are 25.4 mm width×25.4 mm length×0.41 mm thickness. A fourth cycle secant modulus at 10% strain is defined as the slope of the line that intersects the stress-strain curve at 0% and 10% strain for this fourth hysteresis cycle. A minimum of three fabric specimens are measured for each fabric treatment, and the resulting fourth cycle secant moduli are averaged to yield an average fourth cycle secant modulus at 10%. The intrinsic performance of compositions of the present invention are compared by calculating the percentage to which a given composition reduces the fourth cycle secant modulus at 10% strain compared to a control fabric specimen treated with water.

The reported value for average percent RSM is calculated as:

$$100 \% \times \frac{\begin{matrix}{( 4 th cycle secant modulus )}_{CONTROL} - \\ {( 4 th cycle secant modulus )}_{TEST LEG}\end{matrix}}{{( 4 th cycle secant modulus )}_{CONTROL}}$$

### Reduction in Water Uptake

Reduction in water uptake is a measure of the compostions' capacity to impart through-the-day control to hair. Without being bound by theory it is believed that water uptake by the hair leads to a loss in the hair's style and “frizz” so that a reduction in water uptake will be perceived by consumers as improving through-the-day control. Technical benefit was measured via dynamic vapor sorption (DVS) at 25 C.

In the DVS experiment, the hair is first exposed to 0% RH for 30 hours and then the humidity is increased to 90% RH and held constant at 90% RH for 16 hours. Data are reported as the % reduction in water uptake versus a water control, where water uptake is given by total % mass increase of the hair assumed to be water at 90% RH compared to a 0% RH baseline.

TABLE 1
Compositions on fabric comprising select soy oil based silylated oils and select
dimethiconols with and without silica

| Example # | 1-1 | 1-2 | 1-3 | 1-4 | 1-5 | 1-6 | 1-7 | 1-8 | 1-9 | 1-10 | 1-11 | 1-12 | 1-13 | 1-14 | 1-15 |
|---|---|---|---|---|---|---|---|---|---|---|---|---|---|---|---|
| Assumed total oil | 0 | 30 | 30 | 30 | 30 | 30 | 30 | 30 | 30 | 30 | 30 | 30 | 30 | 30 | 30 |
| content | | | | | | | | | | | | | | | |
| Colloidal silica ¹ | 0 | 0 | 0 | 0 | 0 | 0 | 0 | 0 | 0 | 0 | 1.2 | 1.2 | 1.2 | 1.2 | 1.2 |
| 68000 cSt | 0 | 0 | 7.5 | 0 | 0 | 0 | 0 | 0 | 0 | 0 | 0 | 0 | 0 | 0 | 0 |
| dimethiconol | | | | | | | | | | | | | | | |
| emulsion (as | | | | | | | | | | | | | | | |
| active silicone) ² | | | | | | | | | | | | | | | |
| 1000000 cSt | 0 | 0 | 0 | 7.5 | 15 | 0 | 0 | 7.5 | 15 | 15 | 15 | 0 | 0 | 7.5 | 15 |
| dimethiconol | | | | | | | | | | | | | | | |
| emulsion (as | | | | | | | | | | | | | | | |
| active silicone) ³ | | | | | | | | | | | | | | | |
| 2000000 cSt | 0 | 0 | 0 | 0 | 0 | 7.5 | 15 | 0 | 0 | 0 | 0 | 7.5 | 15 | 0 | 0 |
| dimethiconol | | | | | | | | | | | | | | | |
| emulsion (as | | | | | | | | | | | | | | | |
| active silicone) ⁴ | | | | | | | | | | | | | | | |
| silylated soy with | 0 | 30 | 22.5 | 22.5 | 15 | 22.5 | 15 | 0 | 0 | 0 | 15 | 22.5 | 15 | 0 | 0 |
| an average of 0.7 | | | | | | | | | | | | | | | |
| hydrolysable silyl | | | | | | | | | | | | | | | |
| groups | | | | | | | | | | | | | | | |
| silylated soy with | 0 | 0 | 0 | 0 | 0 | 0 | 0 | 22.5 | 15 | 0 | 0 | 0 | 0 | 22.5 | 15 |
| an average of 1.5 | | | | | | | | | | | | | | | |
| hydrolysable silyl | | | | | | | | | | | | | | | |
| groups | | | | | | | | | | | | | | | |
| silylated soy with | 0 | 0 | 0 | 0 | 0 | 0 | 0 | 0 | 0 | 15 | 0 | 0 | 0 | 0 | 0 |
| an average of 2.4 | | | | | | | | | | | | | | | |
| hydrolysable silyl | | | | | | | | | | | | | | | |
| groups | | | | | | | | | | | | | | | |
| Emulsifier ⁵ | 0 | 0.75- | 0.75- | 0.75- | 0.75- | 0.75- | 0.75- | 0.75- | 0.75- | 0.75- | 0.75- | 0.75- | 0.75- | 0.75- | 0.75- |
| | | 7.5 | 7.5 | 7.5 | 7.5 | 7.5 | 7.5 | 7.5 | 7.5 | 7.5 | 7.5 | 7.5 | 7.5 | 7.5 | 7.5 |
| Water | 100 | q.s. | q.s. | q.s. | q.s. | q.s. | q.s. | q.s. | q.s. | q.s. | q.s. | q.s. | q.s. | q.s. | q.s. |
| Avg Time to | 0 | 1 | 28 | >60 | 0 | >60 | >60 | 57 | 49 | 34.3 | >60 | >60 | >60 | >60 | >60 |
| Wick (min) | | | | | | | | | | | | | | | |
| 10 mg/g | | | | | | | | | | | | | | | |
| Avg Time to | 0 | 0.3 | 55 | 55 | 18 | >60 | >60 | 36 | 39 | 0 | 54 | >60 | >60 | 58 | 39 |
| Wick (min) | | | | | | | | | | | | | | | |
| 3 mg/g | | | | | | | | | | | | | | | |
| avg % reduction | 0 | 22.8 | 34.2 | 38.1 | 36.7 | 44.4 | 55.2 | 33.5 | 34.7 | 31.1 | 32.1 | 29.1 | 54.0 | 24.9 | 36.1 |
| in secant | | | | | | | | | | | | | | | |
| modulus, 3 mg/g | | | | | | | | | | | | | | | |

¹ Available as Nalco 1115 from Nalco, Naperville, IL. Weight percent reported as % active silica.
² Sourced as tradename DC1872 from Dow Corning, Midland, MI. Weight percent listed as % active dimethiconol.
³ Prepared by emulsion polymerization of silanol-terminated dimethylsiloxane oligomers, available from Gelest, Morrisville, PA, with dodecylbenzene sulfonic acid, available from Sigma Aldrich, St. Louis, MO. Weight percent listed as % active dimethiconol.
⁴ Sourced as tradename MEM-1788 from Xiameter (a subsidiary of Dow Corning, Midland, MI). Weight percent listed as % active dimethiconol.
⁵ Sodium dodecyl sulfate, available from Sigma Aldrich, St. Louis, MO.

TABLE 2
Compositions on hair

| Example # | 2-1 | 2-2 | 2-3 | 2-4 |
|---|---|---|---|---|
| Total oil content | 0 | 30 | 30 | 30 |
| 68000 cSt dimethiconol (as active | 0 | 0 | 7.5 | 0 |
| silicone) ¹ | | | | |
| 2000000 cSt dimethiconol (as active | 0 | 0 | 0 | 7.5 |
| silicone) ² | | | | |
| silylated soy with an average of 0.7 | 0 | 30 | 22.5 | 22.5 |
| hydrolysable silyl groups | | | | |
| Emulsifiers ⁴ | 0 | 0.75-7.5 | 0.75-7.5 | 0.75-7.5 |
| Water | 100 | q.s. | q.s. | q.s. |
| Average % reduction in water uptake, | 0 | 1.7 | 1.7 | 1.7 |
| 10 mg/g | | | | |

¹ Sourced as tradename DC1872 from Dow Corning, Midland, MI. Weight percent listed as % active dimethiconol.
² Sourced as tradename MEM-1788 from Xiameter (a subsidiary of Dow Corning, Midland, MI). Weight percent listed as % active dimethiconol.
³ Sodium dodecyl sulfate, available from Sigma Aldrich, St. Louis, MO.

TABLE 3
Compositions on fabric comprising select triglyceride silylated oils with and without silica

| Example # | 3-1 | 3-2 | 3-3 | 3-4 | 3-5 | 3-6 | 3-7 | 3-8 | 3-9 | 3-10 | 3-11 |
|---|---|---|---|---|---|---|---|---|---|---|---|
| Total oil content | 30 | 30 | 30 | 30 | 30 | 30 | 30 | 30 | 30 | 30 | 30 |
| Colloidal silica ¹ | 0 | 0 | 0 | 0 | 0 | 1.2 | 1.2 | 1.2 | 1.2 | 1.2 | 1.2 |
| 2000000 cSt dimethiconol (as | 15 | 15 | 15 | 15 | 15 | 15 | 15 | 15 | 15 | 15 | 15 |
| active silicone) ² | | | | | | | | | | | |
| silylated Abyssinian oil with an | 15 | 0 | 0 | 0 | 0 | 15 | 0 | 0 | 0 | 0 | 0 |
| average of 0.8 hydrolysable silyl | | | | | | | | | | | |
| groups | | | | | | | | | | | |
| silylated Abyssinian oil with an | 0 | 15 | 0 | 0 | 0 | 0 | 15 | 0 | 0 | 0 | 0 |
| average of 1.3 hydrolysable silyl | | | | | | | | | | | |
| groups | | | | | | | | | | | |
| silylated high oleic soybean oil | 0 | 0 | 15 | 0 | 0 | 0 | 0 | 15 | 0 | 0 | 0 |
| with an average of 0.8 | | | | | | | | | | | |
| hydrolysable silyl groups | | | | | | | | | | | |
| silylated high oleic soybean oil | 0 | 0 | 0 | 15 | 0 | 0 | 0 | 0 | 15 | 0 | 0 |
| with an average of 1.7 | | | | | | | | | | | |
| hydrolysable silyl groups | | | | | | | | | | | |
| silylated canola oil with an | 0 | 0 | 0 | 0 | 0 | 0 | 0 | 0 | 0 | 15 | 0 |
| average of 0.9 hydrolysable silyl | | | | | | | | | | | |
| groups | | | | | | | | | | | |
| silylated canola oil with an | 0 | 0 | 0 | 0 | 15 | 0 | 0 | 0 | 0 | 0 | 15 |
| average of 1.4 hydrolysable silyl | | | | | | | | | | | |
| groups | | | | | | | | | | | |
| Emulsifiers ³ | 0.75- | 0.75- | 0.75- | 0.75- | 0.75- | 0.75- | 0.75- | 0.75- | 0.75- | 0.75- | 0.75- |
| | 7.5 | 7.5 | 7.5 | 7.5 | 7.5 | 7.5 | 7.5 | 7.5 | 7.5 | 7.5 | 7.5 |
| Water | q.s. | q.s. | q.s. | q.s. | q.s. | q.s. | q.s. | q.s. | q.s. | q.s. | q.s. |
| Avg Time to Wick (min) 10 mg/g | 15.7 | 40.3 | 4.8 | 23.4 | 28.5 | 53.7 | 48.4 | 36.5 | 48.6 | 25.6 | 20.4 |
| avg % reduction in secant | — | 51.0 | 40.7 | 39.4 | 43.0 | 32.7 | 38.3 | 31.7 | 35.0 | 33.8 | 32.4 |
| modulus, 3 mg/g | | | | | | | | | | | |

¹ Available as Nalco 1115 from Nalco, Naperville, IL. Weight percent reported as % active silica.
² Sourced as tradename MEM-1788 from Xiameter (a subsidiary of Dow Corning, Midland, MI). Weight percent listed as % active dimethiconol.
³ Sodium dodecyl sulfate, available from Sigma Aldrich, St. Louis, MO

TABLE 4
Compositions on fabric comprising select particulate benefit agents

| Example # | 4-1 | 4-2 | 4-3 | 4-4 | 4-5 | 4-6 |
|---|---|---|---|---|---|---|
| Total oil content | 0 | 30 | 30 | 30 | 30 | 30 |
| colloidal Silica ¹ | 0 | 0 | 0 | 0 | 0 | 0 |
| Titanium Dioxide ² | 0 | 0 | 0 | 0.5 | 0 | 0 |
| Timiron Silk Gold (TiO2 and | 0 | 0 | 0 | 0 | 0.5 | 0 |
| Mica) ³ | | | | | | |
| Reflecks Dimensions | 0 | 0 | 0 | 0 | 0 | 0.5 |
| shimmering blue pigment ⁴ | | | | | | |
| Belsil DM5500E (as active | 0 | 30 | 0 | 15 | 15 | 15 |
| silicone) ⁵ | | | | | | |
| silylated Abyssinian oil with an | 0 | 0 | 30 | 15 | 15 | 15 |
| average of 1.3 hydrolysable silyl | | | | | | |
| groups | | | | | | |
| Emulsifiers ⁶ | 0 | 0.75- | 0.75- | 0.75- | 0.75- | 0.75- |
| | | 7.5 | 7.5 | 7.5 | 7.5 | 7.5 |
| Water | 100 | q.s. | q.s. | q.s. | q.s. | q.s. |
| Avg Time to Wick (min) 3 mg/g | 0 | 0 | 0.02 | 6.2 | 14.5 | 9.3 |

¹ Available as Syton HT-50 from Sigma Aldrich, St. Louis, MO
² Available as AFDC200 from Kobo Products, Inc., South Plainfield, NJ
³ Available from EMD Chemicals, Philadelphia, PA
⁴ Available from BASF, Iselin, NJ
⁵ Available from Wacker Silicones. Weight percent listed as % active dimethiconol
⁶ Emulsifiers used included Tween 80 and Span 80, available from Sigma Aldrich, St. Louis, MO

TABLE 5
Compositions on fabric comprising select
hydroxyl functional organic species

| Example # | 5-1 | 5-2 | 5-3 |
|---|---|---|---|
| Assumed total oil content | 0 | 10 | 16 |
| PEG 6000¹ | 0 | 5 | 0 |
| guar hydroxypropyl trimonium chloride² | 0 | 0 | 1 |
| silylated Abyssinian oil with an average of 1.3 | 0 | 5 | 15 |
| hydrolysable silyl groups | | | |
| Emulsifiers³ | 0 | 0.75-7.5 | 0.75-7.5 |
| Water | 100 | q.s. | q.s. |
| avg % reduction in secant modulus, 3 mg/g | 0 | 37.6 | 24.0 |

¹Available from Sigma Aldrich, St. Louis, MO
²Available as NHance CG-17 from Ashland Inc., Wilmington, DE
³Emulsifiers used included Tween 80 and Span 80, available from Sigma Aldrich, St. Louis, MO

### Examples

### Consumer Products

### Example 11

Shampoo—A shampoo composition is prepared by conventional methods from the following components.

| | EXAMPLE COMPOSITION | | | | | |
|---|---|---|---|---|---|---|
| Ingredient | A | B | C | D | E | F |
| Water | to | to | to | to | to | to |
| | 100% | 100% | 100% | 100% | 100% | 100% |
| Polyquaternium 76 ¹ | 0.25 | — | — | 0.25 | — | — |
| Guar, Hydroxypropyl Trimonium | — | 0.25 | — | — | 0.25 | — |
| Chloride ² | | | | | | |
| Polyquaternium 6 ³ | — | — | 0.79 | — | — | 0.79 |
| Sodium Laureth Sulfate (SLE3S) ⁴ | 21.43 | 21.43 | 21.43 | — | — | — |
| Sodium Laureth Sulfate (SLE1S) ⁴ | — | — | — | 10.50 | 10.50 | 10.50 |
| Sodium Lauryl Sulfate (SLS) ⁵ | 20.69 | 20.69 | 20.69 | 1.5 | 1.5 | 1.5 |
| Silylated Oil of Examples 1-10, | 0.50 | 0.50 | 1.00 | 0.50 | 0.50 | 1.00 |
| as active wt % silylated oil | | | | | | |
| Cocoamidopropyl Betaine ⁶ | 3.33 | 3.33 | 3.33 | 1.0 | 1.0 | 1.0 |
| Cocoamide MEA ⁷ | 1.0 | 1.0 | 1.0 | 1.0 | 1.0 | 1.0 |
| Ethylene Glycol Distearate ⁸ | 1.50 | 1.50 | 1.50 | 1.50 | 1.50 | 1.50 |
| Sodium Chloride ⁹ | 0.25 | 0.25 | 0.25 | 0.25 | 0.25 | 0.25 |
| Fragrance | 0.70 | 0.70 | 0.70 | 0.70 | 0.70 | 0.70 |
| Preservatives, pH adjusters | Up to | Up to | Up to | Up to | Up to | Up to |
| | 1% | 1% | 1% | 1% | 1% | 1% |

¹ Mirapol ® AT-1, Copolymer of Acrylamide(AM) and TRIQUAT, MW = 1,000,000; CD = 1.6 meq./gram; 10% active; Supplier Rhodia
² Jaguar ® C500, MW - 500,000, CD = 0.7, supplier Rhodia
³ Mirapol ® 100S, 31.5% active, supplier Rhodia
⁴ Sodium Laureth Sulfate, 28% active, supplier: P&G
⁵ Sodium Lauryl Sulfate, 29% active supplier: P&G
⁶ Tego ® betaine F-B, 30% active supplier: Goldschmidt Chemicals
⁷ Monamid CMA, 85% active, supplier Goldschmidt Chemical
⁸ Ethylene Glycol Distearate, EGDS Pure, supplier Goldschmidt Chemical
⁹ Sodium Chloride USP (food grade), supplier Morton; note that salt is an adjustable ingredient, higher or lower levels may be added to achieve target viscosity.

### Example 12

Conditioner examples—A conditioner composition is prepared by conventional methods from the following components.

| | EXAMPLE | |
|---|---|---|
| | COMPOSITION | |
| Ingredient | A | B |
| Water | q.s. to 100% | q.s. to 100% |
| Silylated Oil of Examples 1-10, as active | 1.0 | — |
| wt % silylated oil ¹ | | |
| Silylated Oil of Examples 1-10, as active | — | 1.0 |
| wt % silylated oil ² | | |
| Cyclopentasiloxane ³ | — | 0.61 |
| Behenyl trimethyl ammonium chloride ⁴ | 2.25 | 2.25 |
| Isopropyl alcohol | 0.60 | 0.60 |
| Cetyl alcohol ⁵ | 1.86 | 1.86 |
| Stearyl alcohol ⁶ | 4.64 | 4.64 |
| Disodium EDTA | 0.13 | 0.13 |
| NaOH | 0.01 | 0.01 |
| Benzyl alcohol | 0.40 | 0.40 |
| Methylchloroisothiazolinone/ | 0.0005 | 0.0005 |
| Methylisothiazolinone ⁷ | | |
| Panthenol ⁸ | 0.10 | 0.10 |
| Panthenyl ethyl ether ⁹ | 0.05 | 0.05 |
| Fragrance | 0.35 | 0.35 |

¹ Silylated Oil of Example 1-10, as active wt % silylated oil (mixtures thereof may also be used)
² Silylated Oil of Example 1-10, as active wt % silylated oil (mixtures thereof may also be used)
³ Cyclopentasiloxane: SF1202 available from Momentive Performance Chemicals
⁴ Behenyl trimethyl ammonium chloride/Isopropyl alcohol: Genamin TM KMP available from Clariant
⁵ Cetyl alcohol: Konol TM series available from Shin Nihon Rika
⁶ Stearyl alcohol: Konol TM series available from Shin Nihon Rika
⁷ Methylchloroisothiazolinone/Methylisothiazolinone: Kathon TM CG available from Rohm & Haas
⁸ Panthenol: Available from Roche
⁹ Panthenyl ethyl ether: Available from Roche

### Example 13

Moisturizing oil-in-water skin lotions/creams

| | | | | |
|---|---|---|---|---|
| A | B | C | D | E |

Water Phase:

| | | | | | |
|---|---|---|---|---|---|
| Water | q.s. | q.s. | q.s. | q.s. | q.s. |
| Glycerin | 3 | 5 | 7 | 10 | 15 |
| Disodium EDTA | 0.1 | 0.1 | 0.05 | 0.1 | 0.1 |
| Methylparaben | 0.1 | 0.1 | 0.1 | 0.1 | 0.1 |
| Niacinamide | 2 | 0.5 | — | 3 | 5 |
| Triethanolamine | — | 0.25 | — | — | — |
| D-panthenol | 0.5 | 0.1 | — | 0.5 | 1.5 |
| Sodium Dehydroacetate | 0.5 | 0.1 | 0.5 | 0.1 | 0.5 |
| Benzyl alcohol as fragrance | 0.25 | 0.25 | 0.25 | 0.25 | 0.25 |
| GLW75CAP-MP (75% aq. TiO2 | — | 0.5 | 0.5 | — | — |
| dispersion)¹ | | | | | |
| Hexamidine diisethionate | — | 0.1 | — | — | — |
| Palmitoyl-dipeptide² | 0.00055 | 0.00055 | 0.0001 | 0.00055 | 0.00055 |
| N-acetyl glucosamine | 2 | 1 | 2 | 2 | 1 |
| Soy Isoflavone | 0.5 | — | — | — | — |

Oil Phase:

| | | | | | |
|---|---|---|---|---|---|
| Salicylic Acid | — | — | 1.5 | — | — |
| Isohexadecane | 3 | 3 | 3 | 4 | 3 |
| PPG15 Stearyl Ether | — | — | 4 | — | — |
| Isopropyl Isostearate | 1 | 0.5 | 1.3 | 1.5 | 1.3 |
| Sucrose polyester | 0.7 | — | 0.7 | 1 | 0.7 |
| Dipalmitoylhydroxyproline | — | — | — | 1.0 | — |
| Undecylenoyl Phenylalanine | — | 0.5 | — | — | — |
| Phytosterol | — | — | 0.5 | — | 1.0 |
| Cetyl alcohol | 0.4 | 0.3 | 0.4 | 0.5 | 0.4 |
| Stearyl alcohol | 0.5 | 0.35 | 0.5 | 0.6 | 0.5 |
| Behenyl alcohol | 0.4 | 0.3 | 0.4 | 0.5 | 0.4 |
| PEG-100 stearate | 0.1 | 0.1 | 0.1 | 0.2 | 0.1 |
| Cetearyl glucoside | 0.1 | 0.1 | 0.1 | 0.25 | 0.1 |

Thickener:

| | | | | | |
|---|---|---|---|---|---|
| Polyacrylamide/C₁₃₋₁₄ | 1.5 | — | 2 | 2.5 | 2 |
| isoparaffin/laureth-7 | | | | | |
| Sodium acrylate/sodium | — | 3 | — | — | — |
| acryloyldimethyl taurate | | | | | |
| copolymer/isohexadecane/ | | | | | |
| polysorbate 80 | | | | | |

Additional Ingredients:

| | | | | | |
|---|---|---|---|---|---|
| Silylated Oil of Example 1-10, | 3 | 1 | 2 | 0.5 | 2 |
| as active wt % silylated oil | | | | | |
| Polymethylsilsequioxane | — | — | 0.25 | — | 1 |
| Nylon-12 | — | 0.5 | — | — | — |
| Prestige Silk Violet³ | — | — | — | — | 1 |
| Timiron Splendid Red⁴ | — | 1.0 | — | 2 | — |

¹Available from Kobo products
²Palmitoyl-lysine-threonine available from Sederma
³Titanium dioxide coated mica violet interference pigment available from Eckart
⁴Silica and titanium dioxide coated mica red interference pigment available from Rona

In a suitable vessel, combine the water phase ingredients and heat to 75° C. In a separate suitable vessel, combine the oil phase ingredients and heat to 75° C. Next, add the oil phase to the water phase and mill the resulting emulsion (e.g., with a Tekmar T-25). Then, add the thickener to the emulsion and cool the emulsion to 45° C. while stiffing. At 45° C., add the remaining ingredients. Cool the product and stir to 30° C. and pour into suitable containers.

### Example 14

An antiperspirant soft solid/cream is prepared by conventional methods from the following components.

| | Example | | | |
|---|---|---|---|---|
| Component | A | B | C | D |
| Al Zr Trichlorohydrex Glycinate | 25.25 | 25.25 | 25.25 | 25.25 |
| (solid) | | | | |
| Dimethicone (10 cs) | 5.00 | 5.00 | 5.00 | 5.00 |
| Fully Hydrogenated High Erucic | 5.00 | 5.00 | 5.00 | 5.00 |
| Acid Rapeseed oil (HEAR oil) | | | | |
| Agmatine | 2.50 | 2.50 | 2.50 | 2.50 |
| C-18-36 Acid Triglyceride | 1.25 | 1.25 | 1.25 | 1.25 |
| Syncrowax HGLC | | | | |
| Perfume | 0.75 | 0.75 | 0.75 | 0.75 |
| Calcium Pantothenate (solid) | 0.50 | 0 | 3.50 | 0 |
| BHT | 0.50 | 0.50 | 0.50 | 0.50 |
| Tocopherol Acetate | 0.50 | 0 | 0.50 | 0 |
| Silylated Oil of Example 1-7 | q.s. | q.s. | q.s. | q.s. |
| Total | 100.00 | 100.00 | 100.00 | 100.00 |

### Example 15

### Shave Preparation Composition

| Example | A | B | C | D | E |
|---|---|---|---|---|---|
| Sorbitol 70% Solution | 0.9600% | 0.9615% | 0.9715% | 0.9700% | 0.9715% |
| Glycerin | 4.8000% | 4.8075% | 0.4857% | 0.4850% | 0.4857% |
| hydroxyethyl cellulose¹ | 0.4800% | 0.3846% | 0.4857% | 0.7275% | 0.4857% |
| PEG-90M² | 0.1632% | 0.0577% | 0.1652% | 0.1067% | 0.1652% |
| PEG-23M³ | 0.0480% | 0.0865% | 0.0486% | 0.0582% | 0.0486% |
| PTFE⁴ | 0.1440% | 0.0481% | 0.1457% | 0.1940% | 0.1457% |
| Palmitic acid | 7.4400% | 7.4516% | 7.5291% | 6.3923% | 7.5291% |
| Stearic Acid | 2.4960% | 2.4999% | 2.5259% | 2.1437% | 2.5259% |
| Glyceryl Oleate | 1.3920% | 2.8845% | 1.9430% | 2.4250% | 1.9430% |
| Triethanolamine (99%) | 6.0960% | 6.1055% | 5.8776% | 5.2380% | 6.1690% |
| Lubrajel Oil⁵ | 0.9600% | 0.7211% | 0.9715% | 1.2125% | 0.9715% |
| Fragrance | 1.2960% | 0.7692% | 1.0687% | 0.9700% | 1.3115% |
| Dye | 0.0029% | 0.0025% | 0.0008% | | |
| Menthol | | 0.0481% | | 0.0970% | 0.2429% |
| Silylated Oil of Example 1-10, | 7.2000% | 3.8460% | 9.7150% | 9.7000% | 6.8005% |
| as active wt % silylated oil | | | | | |
| Expancel⁶ | | | | 1.9400% | |
| Dimethicone⁷ | 1.9200% | 3.8460% | 1.9430% | | 2.9145% |
| Iso E Super⁸ | 0.0576% | 0.1923% | 0.0583% | 0.0582% | 0.1457% |
| PPG-15 Stearyl Ether⁹ | 0.2400% | 0.3365% | | 0.2425% | 0.3400% |
| Isopentane (and) Isobutane | 4.0000% | 0.9615% | 2.8500% | 3.0000% | 2.8500% |
| Water | q.s to | q.s to | q.s to | q.s to | q.s to |
| | 100% | 100% | 100% | 100% | 100% |
| Example | F | G | H | I | J |
| Sorbitol 70% Solution | 0.9600% | 0.9600% | 0.9725% | 0.9625% | 0.9715% |
| Glycerin | 0.4800% | 0.4800% | 0.4863% | 9.6250% | 0.4857% |
| hydroxyethyl cellulose¹ | 0.4800% | 0.4800% | 0.4863% | 0.4813% | 0.4857% |
| PEG-90M² | 0.1632% | 0.1632% | 0.1653% | 0.1636% | 0.1652% |
| PEG-23M³ | 0.0480% | 0.0480% | 0.0584% | 0.0578% | |
| PTFE⁴ | 0.1440% | 0.1440% | 0.1459% | 0.1444% | |
| Palmitic acid | 7.4400% | 7.4400% | 9.0443% | 7.4594% | 7.5291% |
| Stearic Acid | 2.4960% | 2.4960% | 3.0342% | 2.5025% | 2.5259% |
| Glyceryl Oleate | 1.7280% | 1.7280% | 0.9725% | 1.7325% | 1.9430% |
| Triethanolamine (99%) | 6.0960% | 6.0960% | 7.4105% | 6.1119% | 6.1690% |
| Lubrajel Oil⁵ | 0.9600% | 0.9600% | | 0.9625% | |
| Fragrance | 1.2960% | 1.2960% | 0.5835% | 1.2994% | 0.7692% |
| Dye | 0.0008% | | 0.0022% | | 0.0022% |
| Menthol | 0.2208% | 0.2208% | | | 0.2429% |
| Silylated Oil of Example 1-10, | 4.8000% | 4.8000% | 7.7800% | 4.8125% | 7.7800% |
| as active wt % silylated oil | | | | | |
| Expancel⁶ | 0.9600% | 0.9600% | | 0.9625% | |
| Dimethicone⁷ | 2.8800% | 2.8800% | | 1.9250% | 2.9145% |
| Iso E Super⁸ | 0.1440% | 0.1440% | | 0.1925% | |
| PPG-15 Stearyl Ether⁹ | 0.3360% | 0.3360% | | 0.3369% | 0.3400% |
| Isopentane (and) Isobutane | 4.0000% | 4.0000% | 2.7500% | 3.7500% | 2.8500% |
| Water | q.s to | q.s to | q.s to | q.s to | q.s to |
| | 100% | 100% | 100% | 100% | 100% |

¹Available as Natrosol 250 HHR from Hercules Inc., Wilmington, DE
²Available as Polyox WSR-301 from Amerchol Corp., Piscataway, NJ
³Available as Polyox WSR N-12K from Amerchol Corp., Piscataway, NJ
⁴Available as Microslip 519 from Micro Powders Inc., Tarrytown, NY
⁵Available from Guardian Laboratories, Hauppauge, NY
⁶Available from AkzoNobel, Bridgewter, NJ
⁷Available as Xiameter(R) PMX-200 Silicone Fluid from Dow Corning Corp., Midland, MI
⁸Available from International Flavors & Fragrances Inc., Shrewsbury, NJ
⁹Available as Arlamol PS15E from Croda, Inc., Edison, NJ

### Example 16

### Heavy Duty Liquid Laundry Detergent Compositions

| | A | B | C | D | E | F |
|---|---|---|---|---|---|---|
| | (wt %) | (wt %) | (wt %) | (wt %) | (wt %) | (wt %) |
| AES C₁₂₋₁₅ alkyl ethoxy (1.8) sulfate | 11 | 10 | 4 | 6.32 | 0 | 0 |
| AE3S | 0 | 0 | 0 | 0 | 2.4 | 0 |
| Linear alkyl benzene sulfonate | 1.4 | 4 | 8 | 3.3 | 5 | 8 |
| HSAS | 3 | 5.1 | 3 | 0 | 0 | 0 |
| Sodium formate | 1.6 | 0.09 | 1.2 | 0.04 | 1.6 | 1.2 |
| Sodium hydroxide | 2.3 | 3.8 | 1.7 | 1.9 | 1.7 | 2.5 |
| Monoethanolamine | 1.4 | 1.49 | 1.0 | 0.7 | 0 | 0 |
| Diethylene glycol | 5.5 | 0.0 | 4.1 | 0.0 | 0 | 0 |
| AE9 | 0.4 | 0.6 | 0.3 | 0.3 | 0 | 0 |
| AE7 | 0 | 0 | 0 | 0 | 2.4 | 6 |
| Chelant | 0.15 | 0.15 | 0.11 | 0.07 | 0.5 | 0.11 |
| Citric Acid | 2.5 | 3.96 | 1.88 | 1.98 | 0.9 | 2.5 |
| C₁₂₋₁₄ dimethyl Amine Oxide | 0.3 | 0.73 | 0.23 | 0.37 | 0 | 0 |
| C₁₂₋₁₈ Fatty Acid | 0.8 | 1.9 | 0.6 | 0.99 | 1.2 | 0 |
| 4-formyl-phenylboronic acid | 0 | 0 | 0 | 0 | 0.05 | 0.02 |
| Borax | 1.43 | 1.5 | 1.1 | 0.75 | 0 | 1.07 |
| Ethanol | 1.54 | 1.77 | 1.15 | 0.89 | 0 | 3 |
| Ethoxylated (EO₁₅) tetraethylene pentamine | 0.3 | 0.33 | 0.23 | 0.17 | 0.0 | 0.0 |
| Ethoxylated hexamethylene diamine | 0.8 | 0.81 | 0.6 | 0.4 | 1 | 1 |
| 1,2-Propanediol | 0.0 | 6.6 | 0.0 | 3.3 | 0.5 | 2 |
| Protease (40.6 mg active/g) | 0.8 | 0.6 | 0.7 | 0.9 | 0.7 | 0.6 |
| Mannanase: Mannaway ® (25 mg active/g) | 0.07 | 0.05 | 0.045 | 0.06 | 0.04 | 0.045 |
| Amylase: Stainzyme ® (15 mg active/g) | 0.3 | 0 | 0.3 | 0.1 | 0 | 0.4 |
| Amylase: Natalase ® (29 mg active/g) | 0 | 0.2 | 0.1 | 0.15 | 0.07 | 0 |
| Lipex ® (18 mg active/g) | 0.4 | 0.2 | 0.3 | 0.1 | 0.2 | 0 |
| Silylated oil of any of Examples 1-10, as active | 3.0 | 3.0 | 3.0 | 3.0 | 3.0 | 3.0 |
| wt % oil | | | | | | |
| Liquitint ® Violet CT (active) | 0.006 | 0.002 | 0 | 0 | 0 | 0.002 |

| | |
|---|---|
| Water, perfume, dyes & other components | Balance |

### Example 17

### Laundry Detergent

| | (wt %) |
|---|---|
| Alkylbenzene sulfonic acid | 21.0 |
| C₁₄₋₁₅ alkyl 8-ethoxylate | 18.0 |
| C₁₂₋₁₈ Fatty acid | 15.0 |
| Protease (40.6 mg active/g)** | 1.5 |
| Natalase ® (29 mg active/g)** | 0.2 |
| Mannanase (Mannaway ®, 11 mg active/g)** | 0.1 |
| Xyloglucanase (Whitezyme ®, 20 mg active/g)** | 0.2 |
| Silylated oil of any of Examples 1-10, as active wt % of oil | 1.0 |
| A compound having the following general structure: | 2.0 |
| bis((C₂H₅O)(C₂H₄O)n)(CH₃)—N⁺—CₓH₂ₓ—N⁺— (CH₃)- | |
| bis((C₂H₅O)(C₂H₄O)n), wherein n = from 20 to 30, and x = | |
| from 3 to 8, or sulphated or sulphonated variants thereof | |
| Ethoxylated Polyethylenimine ² | 0.8 |
| Hydroxyethane diphosphonate (HEDP) | 0.8 |
| Fluorescent Brightener 1 | 0.2 |
| Solvents (1,2 propanediol, ethanol), stabilizers | 15.0 |
| Hydrogenated castor oil derivative structurant | 0.1 |
| Perfume | 1.6 |
| Core Shell Melamine-formaldehyde encapsulate of | 0.10 |
| perfume | |
| Ethoxylated thiophene Hueing Dye | 0.004 |
| Buffers (sodium hydroxide, Monoethanolamine) | To pH 8.2 |
| Water* and minors (antifoam, aesthetics) | To 100% |

*Based on total cleaning and/or treatment composition weight, a total of no more than 7% water
¹ Random graft copolymer is a polyvinyl acetate grafted polyethylene oxide copolymer having a polyethylene oxide backbone and multiple polyvinyl acetate side chains. The molecular weight of the polyethylene oxide backbone is about 6000 and the weight ratio of the polyethylene oxide to polyvinyl acetate is about 40 to 60 and no more than 1 grafting point per 50 ethylene oxide units.
² Polyethyleneimine (MW = 600) with 20 ethoxylate groups per —NH.
**Remark: all enzyme levels expressed as % enzyme raw material

### Example 18

Unit Dose compositions—This Example provides various formulations for unit dose laundry detergents. Such unit dose formulations can comprise one or multiple compartments. The following unit dose laundry detergent formulations of the present invention are provided below.

Unit Dose Compositions

| Ingredients | A | B | C | D | E |
|---|---|---|---|---|---|
| Alkylbenzene sulfonic acid C 11-13, | 14.5 | 14.5 | 14.5 | 14.5 | 14.5 |
| 23.5% 2-phenyl isomer | | | | | |
| C₁₂₋₁₄ alkyl ethoxy 3 sulfate | 7.5 | 7.5 | 7.5 | 7.5 | 7.5 |
| C₁₂₋₁₄ alkyl 7-ethoxylate | 13.0 | 13.0 | 13.0 | 13.0 | 13.0 |
| Citric Acid | 0.6 | 0.6 | 0.6 | 0.6 | 0.6 |
| Fatty Acid | 14.8 | 14.8 | 14.8 | 14.8 | 14.8 |
| Enzymes (as % raw material not | 1.7 | 1.7 | 1.7 | 1.7 | 1.7 |
| active) | | | | | |
| Protease (as % active) | 0.05 | 0.1 | 0.02 | 0.03 | 0.03 |
| Ethoxylated Polyethylenimine¹ | 4.0 | 4.0 | 4.0 | 4.0 | 4.0 |
| Silylated Oil of Examples 1-10, | 0.2-6 | 0.2-6 | 0.2-6 | 0.2-6 | 0.2-6 |
| as active wt % silylated oil | | | | | |
| Hydroxyethane diphosphonic acid | 1.2 | 1.2 | 1.2 | 1.2 | 1.2 |
| Brightener | 0.3 | 0.3 | 0.3 | 0.3 | 0.3 |
| P-diol | 15.8 | 13.8 | 13.8 | 13.8 | 13.8 |
| Glycerol | 6.1 | 6.1 | 6.1 | 6.1 | 6.1 |
| MEA | 8.0 | 8.0 | 8.0 | 8.0 | 8.0 |
| TIPA | — | — | 2.0 | — | — |
| TEA | — | 2.0 | — | — | — |
| Cumene sulphonate | — | — | — | — | 2.0 |
| cyclohexyl dimethanol | — | — | — | 2.0 | — |
| Water | 10 | 10 | 10 | 10 | 10 |
| Structurant | 0.14 | 0.14 | 0.14 | 0.14 | 0.14 |
| Perfume | 1.9 | 1.9 | 1.9 | 1.9 | 1.9 |

| | |
|---|---|
| Buffers (monoethanolamine) | To pH 8.0 |
| Solvents (1,2 propanediol, ethanol) | To 100% |

¹Polyethylenimine (MW = 600) with 20 ethoxylate groups per —NH.

### Example 19

### Bleach & Laundry Additive Detergent Formulations

| Ingredients | A | B | C | D |
|---|---|---|---|---|
| AES¹ | 6.0 | 15.4 | 16.0 | 10.0 |
| LAS² | 12.0 | 4.6 | — | 26.1 |
| MEA-HSAS³ | — | — | 3.5 | — |
| Silylated oil of any of | 3.0 | 3.0 | 3.0 | 3.0 |
| Examples 1-10, as active | | | | |
| wt % oil | | | | |
| DTPA: Diethylene triamine | — | 1.5 | — | 2.6 |
| pentaacetic acid | | | | |
| 4,5-Dihydroxy-1,3- | — | — | — | 1.4 |
| benzenedisulfonic acid | | | | |
| disodium salt | | | | |
| 1,2-propandiol | 10 | — | — | 15 |
| Copolymer of | | | | |
| dimethylterephthalate, 1,2- | | | | |
| propylene glycol, methyl | | | | |
| capped PEG | | | | |
| Poly(ethyleneimine) | 1.8 | | | |
| ethoxylated, PEI600 E20 | | | | |
| Acrylic acid/maleic acid | | 2.9 | | |
| copolymer | | | | |
| Acusol 880 (Hydrophobically | | | 2.0 | 2.9 |
| Modified Non-Ionic Polyol) | | | | |
| Protease (55 mg/g active)** | — | — | — | 0.1 |
| Amylase (30 mg/g active)** | — | — | — | 0.02 |
| Perfume | 0.2 | 0.03 | 0.17 | 0.15 |
| Brightener | — | — | 0.15 | 0.18 |
| water, other optional | to 100% | to 100% | to 100% | to 100% |
| agents/components* | balance | balance | balance | balance |

¹AES = C₁₀-C₁₈ alkyl ethoxy sulfate supplied by Shell Chemicals.
²LAS = C₉-C₁₅ linear alkyl benzene sulfonate supplied by Huntsman Corp
³HSAS = HC1617HSAS (mid-branched primary alkyl sulfate surfactants having an average carbon chain length of from about 16 to 17)
*Other optional agents/components include suds suppressors, structuring agents such as those based on Hydrogenated Castor Oil (preferably Hydrogenated Castor Oil, Anionic Premix), solvents and/or Mica pearlescent aesthetic enhancer.
**Remark: all enzyme levels expressed as % enzyme raw material

### Example 20

Rinse-Added Fabric Care Compositions—Rinse-Added fabric care compositions are prepared by mixing together ingredients shown below:

| Ingredient | A | B | C |
|---|---|---|---|
| Fabric Softener Active¹ | 11.0 | 11.0 | 11.0 |
| Quaternized polyacrylamide² | 0.25 | 0.25 | 0.25 |
| Calcium chloride | 0.15 | 0.15 | 0.15 |
| Silylated oil of any of Examples | — | 5.0 | 5.0 |
| 1-10, as active wt % oil | | | |
| Silicone⁴ | — | — | 5.0 |
| Perfume | 1.3 | 1.3 | 1.3 |
| Perfume microcapsule³ | 0.65 | 0.65 | 0.65 |
| Water, suds suppressor, stabilizers, | to 100% | to 100% | to 100% |
| pH control agents, buffers, dyes & | pH = 3.0 | pH = 3.0 | pH = 3.0 |
| other optional ingredients | | | |

¹N,N di(tallowoyloxyethyl)—N,N dimethylammonium chloride available from Evonik Corporation, Hopewell, VA.
²Cationic polyacrylamide polymer such as a copolymer of acrylamide/[2-(acryloylamino)ethyl]tri-methylammonium chloride (quaternized dimethyl aminoethyl acrylate) available from BASF, AG, Ludwigshafen under the trade name Sedipur 544.
³Available from Appleton Paper of Appleton, WI
⁴Silicone or aminosilicone, such as Dimethylsiloxane polymer available from Dow Corning ® Corporation, Midland, MI under the trade name DC-1664, or Aminoethylaminopropylmethylsiloxane available from Shin-Etsu Silicones, Akron, OH under the trade name X-22-86993S

### Example 21

### Rinse-Added Fabric Care Compositions Tested for Through the Rinse Softness/Phabrometer

Without being bound by theory, it is believed that fabric extraction energy is a technical measure of fabric softness. In this test, terry fabrics were run-through an automatic mini-washer with the compositions of Example 21 in the rinse-cycle.

The fabric used in the miniwasher is a white terry cloth hand towel, manufactured by Standard Textile. The brand name is Euro Touch and is composed of 100% cotton. Fabrics are cut in half to yield a weight of 50-60 grams and desized using standard procedures. Four hand towel halves were combined with additional 100% cotton ballast to yield a total fabric weight of 250-300 grams per miniwasher. Fabrics were first washed with a 5.84 g dose of Tide Free & Gentle laundry detergent in 2 gal of 6 GPG (GPG=hardness grains per gallon) water. During the rinse cycle, 2.4 g of the rinse added fabric treatment was added. Upon completion of the rinse and spin cycles, fabrics were tumble dried. A set of reference fabrics were prepared washed with a 5.84 g dose of Tide Free & Gentle laundry detergent in 2 gal of 6 GPG (GPG=hardness grains per gallon) water where no rinse added fabric treatment was added. Upon completion of the rinse and spin cycles, fabrics were tumble dried. For each treatment including the reference fabrics, a total of three wash-rinse-dry cycles were completed.

Extraction energy is measured using a Phabrometer Fabric Evaluation System, manufactured by Nu Cybertek, Inc, Davis, Calif. Treated fabrics are cut into 11 cm diameter circles and equilibrated in a constant temperature (CT) room for 24 hours before measuring. The CT room temperature is 20-25 deg. C. with a relative humidity of 50%. A fabric circle is placed between 2 rings. The top ring is weighted and can be varied based on fabric type. A small probe pushes the fabric through the hole in the ring (perpendicular to the fabric surface). The instrument records the force (as voltage) needed to push the fabric through the ring as a function of time. Between each fabric measurement, the bottom of the weight, the inside of the ring, and the base in which the ring is sitting are cleaned with an alcohol wipe having 70% isopropyl alcohol and 30% deionized water. Alcohol wipes were purchased from VWR International. All raw data is exported to Microsoft Excel. There are 108 data points in each exported curve, but only the first 85 are used. Each curve is integrated from 1 to 85 and the sum is reported as the unitless “Extraction Energy”. For each test treatment a minimum of 8 fabric circles are evaluated (two circles from each of four terry cloths) and a sample Standard Deviation is calculated. “Extraction Energy Reduction” (EER) is obtained by subtraction the extraction energy average of the fabric samples treated with test legs in the table below from the average extraction energy of the control sample. Without being bound by theory, a higher EER indicates more softening performance.

Rinse-Added fabric care compositions are prepared by mixing together ingredients shown below:

| Ingredient | A | B | C |
|---|---|---|---|
| Fabric Softener Active¹ | 11.0 | 11.0 | 11.0 |
| Quaternized polyacrylamide² | 0.175 | 0.175 | 0.175 |
| Calcium chloride | 0.15 | 0.15 | 0.15 |
| Brij O2 | 0.33 | 0.33 | 0.33 |
| Brij O10 | 0.05 | 0.05 | 0.05 |
| silylated soy with an average of 0.7 | 1.5 | 1.5 | 1.5 |
| hydrolysable silyl groups (wt % as | | | |
| active silylated oil)³ | | | |
| Perfume | 1.5 | 1.5 | 1.5 |
| Perfume microcapsule⁴ | 0.33 | 0.33 | 0.33 |
| Dimethiconol (wt % as active | 0 | 1.5 | 1.5 |
| silicone)⁵ | | | |
| Colloidal Silica⁶ | 0 | 0 | 0.06 |
| Water soluble dialkyl quat⁷ | 0.25 | 0.25 | 0.25 |
| Water, suds suppressor, stabilizers, | to 100% | to 100% | to 100% |
| pH control agents, buffers, dyes & | pH = 3.0 | pH = 3.0 | pH = 3.0 |
| other optional ingredients* | | | |
| Reduction in Extraction energy | 7.06 | 8.94 | 6.95 |
| (unitless) | | | |

¹N,N di(tallowoyloxyethyl)—N,N dimethylammonium chloride available from Evonik Corporation, Hopewell, VA.
²Cationic polyacrylamide polymer such as a copolymer of acrylamide/[2-(acryloylamino)ethyl]tri-methylammonium chloride (quaternized dimethyl aminoethyl acrylate) available from BASF, AG, Ludwigshafen under the trade name Sedipur 544.
³Silylated soy was emulsified as a 20 wt % oil emulsion with Brij O2 and Brij O10 prior to adding to composition. Weight percent listed in table is active silylated soybean oil.
⁴Available from Appleton Paper of Appleton, WI
⁵Sourced as an emulsion under tradename MEM-1788 from Xiameter (a subsidiary of Dow Corning, Midland, MI). Weight percent listed as % active dimethiconol.
⁶Available as Nalco 1115 from Nalco, Naperville, IL. Weight percent reported as % active silica.
⁷Didecyl dimethyl ammonium chloride under the trade name Bardac ® 2280 or Hydrogenated tallowalkyl(2-ethylhexyl)dimethyl ammonium methylsulfate from AkzoNobel under the trade name Arquad ® HTL8-MS
*Other optional agents/components include suds suppressors, structuring agents such as those based on Hydrogenated Castor Oil (preferably Hydrogenated Castor Oil, Anionic Premix), dyes, solvents, perfumes and/or aesthetic enhancers.

### Example 22

### Rinse Added Fabric Treatment Tested for Through the Wash Softness/Friction

Without being bound by theory, it is believed that fabric friction is a technical measure of fabric softness. In this test, terry fabrics were run-through an automatic mini-washer with the compositions of Example 22 in the rinse-cycle.

The fabric used in the miniwasher is a white terry cloth hand towel, manufactured by Standard Textile. The brand name is Euro Touch and is composed of 100% cotton. Fabrics are cut in half to yield a weight of 50-60 grams and desized using standard procedures. Four hand towel halves were combined with additional 100% cotton ballast to yield a total fabric weight of 250-300 grams per miniwasher. Fabrics were first washed with a 5.84 g dose of Tide Free & Gentle laundry detergent in 2 gal of 6 GPG (GPG=hardness grains per gallon) water. During the rinse cycle, 4.73 g of the rinse added fabric treatment was added. Upon completion of the rinse and spin cycles, fabrics were tumble dried. A set of reference fabrics were prepared washed with a 5.84 g dose of Tide Free & Gentle laundry detergent in 2 gal of 6 GPG (GPG=hardness grains per gallon) water where no rinse added fabric treatment was added. Upon completion of the rinse and spin cycles, fabrics were tumble dried. For each treatment including the reference fabrics, a total of three wash-rinse-dry cycles were completed.

When drying of the fabrics is completed, all fabric cloths are equilibrated for a minimum of 8 hours at 20-25 deg. C. and 50% Relative Humidity. Treated and equilibrated fabrics are measured within 2 days of treatment. Treated fabrics are laid flat and stacked no more than 10 cloths high while equilibrating. Friction measurements are all conducted under the same environmental conditions use during the conditioning/equilibration step.

A Thwing-Albert FP2250 Friction/Peel Tester with a 2 kilogram force load cell is used to measure fabric to fabric friction. (Thwing Albert Instrument Company, West Berlin, N.J.). The sled is a clamping style sled with a 6.4 by 6.4 cm footprint and weighs 200 grams (Thwing Albert Model Number 00225-218). The distance between the load cell to the sled is set at 10.2 cm. The crosshead arm height to the sample stage is adjusted to 25 mm (measured from the bottom of the cross arm to the top of the stage) to ensure that the sled remains parallel to and in contact with the fabric during the measurement. The 11.4 cm×6.4 cm cut fabric piece is attached to the clamping sled so that the face of the fabric on the sled is pulled across the face of the fabric on the sample plate. The sled is placed on the fabric and attached to the load cell. The crosshead is moved until the load cell registers between ˜1.0-2.0 gf. Then, it is moved back until the load reads 0.0gf. At this point the measurement is made and the Kinetic Coefficient of Friction (kCOF) recorded. For each treatment, at least four replicate fabrics are measured and the results averaged.

| Ingredients* | A | B |
|---|---|---|
| Cationic deposition aid polymer¹ | 0.4 | 0.4 |
| Dimethiconol² | 4.5 | 4.5 |
| Tallow alkyl ethoxylate (TAE 80, approx. 80 molar | 1.0 | 1.0 |
| 0.1 proportions of ethylene oxide) | | |
| Diethylene glycol butyl ether | 4 | 4 |
| Brij O2 | 0.986 | 0.986 |
| Brij O10 | 0.142 | 0.142 |
| silylated soy with an average of 0.7 hydrolysable | 4.5 | 4.5 |
| silyl groups (wt % as active silylated oil)³ | | |
| Colloidal silica⁴ | 0.36 | 0 |
| Glacial Acetic acid | 0.25 | 0.25 |
| water | to 100% | to 100% |
| | balance | balance |
| kinetic coefficient of friction⁵ | 1.399 | 1.292 |

¹Water soluble cationic polymer such as a copolymer of Acrylamide and methacrylamidopropyl trimethyl ammonium chloride (MAPTAC), available from Nalco.
²Sourced as an emulsion under tradename MEM-1788 from Xiameter (a subsidiary of Dow Corning, Midland, MI). Weight percent listed as % active dimethiconol.
³Silylated soy was emulsified as a 20 wt % oil emulsion with Brij O2 and Brij O10 prior to adding to composition. Weight percent listed in table is active silylated soybean oil.
⁴Available as Nalco 1115 from Nalco, Naperville, IL. Weight percent reported as % active silica.
⁵The kinetic coefficient of friction for the water-only control was measured as 1.470
*Other optional agents/components include suds suppressors, structuring agents such as those based on Hydrogenated Castor Oil (preferably Hydrogenated Castor Oil, Anionic Premix), dyes, solvents, perfumes, preservatives, mica pearlescent aesthetic enhancer and/or aesthetic enhancers.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular aspects of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A consumer product comprising:
(a) silane-modified oil comprising:
(i) a hydrocarbon chain selected from the group consisting of: a saturated oil, an unsaturated oil, and mixtures thereof; and
(ii) a hydrolysable silyl group covalently bonded to the hydrocarbon chain;
and
(b) perfume.

2. The consumer product of claim 1, wherein said consumer product composition is selected from the group consisting of a beauty care product, hand washing product, body wash product, shampoo product, conditioner product, cosmetic product, hair removal product, laundry product, laundry rinse additive product, laundry detergent product, hard surface cleaning product, hand dishwashing product, automatic dishwashing product, unit dose form automatic dishwashing or laundry product, nonwoven fabric product, sanitary tissue product, and absorbent article product.

3. The consumer product of claim 1, wherein said silane-modified oil comprises less than about 10%, by weight of said silane-modified oil, of residual reagent comprising silicon.

4. The consumer product of claim 3, wherein said silane-modified oil comprises less than about 0.1%, by weight of said silane-modified oil, of residual reagent comprising silicon.

5. The consumer product of claim 1, wherein said oil of said silane-modified oil is a triglyceride oil.

6. The consumer product of claim 1, wherein said oil of said silane-modified oil is soybean oil.

7. The consumer product of claim 1, wherein said silane-modified oil comprises a polymer comprising one or more silanol and/or hydrolysable siloxy residues.

8. The consumer product of claim 7, wherein said polymer is a synthetic polymer made by polymerizing one or more monomers selected from the group consisting of N,N-dialkylaminoalkyl acrylate, N,N-dialkylaminoalkyl methacrylate, N,N-dialkylaminoalkyl acrylamide, N,N-dialkylaminoalkylmethacrylamide, quaternized N,N dialkylaminoalkyl acrylate quaternized N,N-dialkylaminoalkyl methacrylate, quaternized N,N-dialkylaminoalkyl acrylamide, quaternized N,N-dialkylaminoalkylmethacrylamide, Methacryloamidopropyl-pentamethyl-1,3-propylene-2-ol-ammonium dichloride, N,N,N,N′,N′,N″,N″-heptamethyl-N″-3-(1-oxo-2-methyl-2-propenyl)aminopropyl-9-oxo-8-azo-decane-1,4,10-triammonium trichloride, vinylamine and its derivatives, allylamine and its derivatives, vinyl imidazole, quaternized vinyl imidazole and diallyl dialkyl ammonium chloride, N,N-dialkyl acrylamide, methacrylamide, N,N-dialkylmethacrylamide, C₁-C₁₂ alkyl acrylate, C₁-C₁₂ hydroxyalkyl acrylate, polyalkylene glyol acrylate, C₁-C₁₂ alkyl methacrylate, C₁-C₁₂ hydroxyalkyl methacrylate, polyalkylene glycol methacrylate, styrene, butadiene, isoprene, butane, isobutene, vinyl acetate, vinyl alcohol, vinyl formamide, vinyl acetamide, vinyl alkyl ether, vinyl pyridine, vinyl pyrrolidone, vinyl imidazole, vinyl caprolactam, acrylic acid, methacrylic acid, maleic acid, vinyl sulfonic acid, styrene sulfonic acid, acrylamidopropylmethane sulfonic acid (AMPS), salts thereof, and mixtures thereof.

9. The consumer product of claim 7, wherein said polymer is a synthetic polymer made by polymerizing isobutene.

10. The consumer product of claim 7, wherein said polymer has a molecular weight of greater than about 500.

11. The consumer product of claim 7, wherein said polymer has a molecular weight of less than about 8,000.

12. The consumer product of claim 7, wherein said polymer has a molecular weight of from about 500 to about 8,000.

13. The consumer product of claim 1, wherein said silane-modified oil comprises:
(i) fewer than 1.2 hydrolysable silyl groups covalently bonded, on average, per molecule of silane-modified oil;
(ii) more than 5.0 hydrolysable silyl groups covalently bonded, on average, per molecule of silane-modified oil; or
(iii) from about 0.7 to about 2.4 hydrolysable silyl groups covalently bonded, on average, per molecule of silane-modified oil.

14. The consumer product of claim 1, wherein said silane-modified oil is in the form of a particle comprising:
(a) a particle core having an interfacial surface; and
(b) said silane-modified oil attached to said interfacial surface.

15. The consumer product of claim 1, wherein said silane-modified oil is emulsified with one or more surfactant(s).

16. The consumer product of claim 1, wherein said consumer product further comprises a hydroxyl functionalized organic species.

17. The consumer product of claim 16, wherein said hydroxyl functionalized organic species is selected from the group consisting of monosaccharides, disaccharides, oligosaccharides, polysaccharides, functionalized monosaccharides, functionalized disaccharides, functionalized oligosaccharides, functionalized polysaccharides, cellulose, guar, starch, cyclodextrin, hydroxypropyl guar, hydroxypropyl cellulose, guar hydroxypropyltrimonium chloride, polyquarternium-10, organo-silicone material, polymers, vinyl polymers, hydroxyl terminated polybutadiene, glycols, poly-glycols, ethers, poly-ethers, polyalkylene oxides, polyethylene oxide, polypropylene oxide, derivatives thereof, and mixtures thereof.

18. The consumer product of claim 17, wherein said hydroxyl functionalized organic species is an organo-silicone material.

19. The consumer product of claim 18, wherein said hydroxyl functionalized organic species is dimethiconol.

20. The consumer product of claim 1, wherein said consumer product further comprises:
(i) a hydroxyl functionalized inorganic particle;
(ii) a particulate benefit agent;
(iii) a preservative; or
(iv) mixtures thereof.

21. The consumer product of claim 20, wherein said hydroxyl functionalized inorganic particle is selected from the group consisting of metal oxides selected from the group consisting of titania, alumina, and mixtures thereof; metallocenes; zeolites; clays; pigments; and mixtures thereof.

22. The consumer product of claim 20, wherein said particulate benefit agent is selected from the group consisting of pigments, clays, personal care actives, anti-perspirant actives, encapsulated liquid actives, and mixtures thereof.

23. The consumer product of claim 22, wherein said particulate benefit agent is a perfume microcapsule.

24. The consumer product of claim 1, wherein said consumer product comprises a silane-modified, oil-based gel network comprising the reaction product of:
(a) said silane-modified oil:
(b) said hydroxyl functional organic species; and
(c) water;
wherein:
(i) at least some of said hydrolysable silyl groups of said silane-modified oil have been hydrolyzed with said water and condensed, thereby forming covalent intermolecular siloxane crosslinks between silane-modified oil molecules in the crosslinked silane-modified oil; and
(ii) the crosslinked silane-modified oil is sufficiently crosslinked with the intermolecular siloxane crosslinks to form a networked gel; and
(d) a carrier, wherein said carrier is aqueous or non-aqueous.

25. A method for treating a surface, comprising the steps of:
(a) applying a consumer product according to claim 1 to said surface; and
(b) optionally applying water to said surface.

26. The method of claim 25, wherein said surface being treated is selected from the group consisting of fabric, textiles, leather, non-woven substrates, woven substrates, fibers, carpet, upholstery, glass, ceramic, skin, hair, fingernails, stone, masonry, wood, plastic, paper, cardboard, metal, packaging, a packaging component, and combinations thereof.
